(19) United States
(12) Patent Application Publication
(10) Pub. No.: US 2012/0128587 A1
(43) Pub. Date: May 24, 2012

(54) FOLATE-TARGETED DIAGNOSTICS AND TREATMENT

(75) Inventors: Christopher Paul Leamon, West Lafayette, IN (US); Richard Messmann, Brighton, MI (US); David Morgenstern, Indianapolis, IN (US)

(73) Assignee: ENDOCYTE, INC., West Lafayette, IN (US)

(21) Appl. No.: 13/388,184

(22) PCT Filed: Jul. 30, 2010
(86) PCT No.: PCT/US10/43992

§ 371 Date: Jan. 31, 2012

Related U.S. Application Data
(60) Provisional application No. 61/230,595, filed on Jul. 31, 2009.
(60) Provisional application No. 61/346,444, filed on May 19, 2010.
(60) Provisional application No. 61/351,022, filed on Jun. 3, 2010.

(51) Int. Cl. A61K 51/08; A61P 35/04; A61P 35/00; A61K 38/08; A61K 9/127

(52) U.S. Cl. 424/1.69; 514/19.3; 424/450

(57) ABSTRACT

Methods of detecting and assessing functionally active folate receptors on tumors and treatment associated with those tumors are described. Also described are methods of selecting ovarian and lung cancer patients for therapy with a folate-vinca conjugate by identifying functionally active folate receptors on the tumors of the patient. Also described are methods and compositions for treating folate receptor expressing epithelial tumors with a folate-vinca conjugate in combination with doxorubicin such as pegylated liposomal doxorubicin in which the tumors include ovarian, endometrial or non-small cell lung cancer tumors, including platinum-resistant ovarian tumors and platinum sensitive ovarian tumors. Also described are methods of treating platinum-resistant ovarian cancer using a folate-targeted drug, in the absence or presence of selecting the patient by identifying functionally active folate receptors on the tumors of the patient.

This application claims the benefit of U.S. provisional applications 61/230,595, filed 31 Jul. 2009; 61/346,444, filed 19 May 2010; and 61/351,022, filed 3 Jun. 2010, each of which is incorporated herein by reference in its entirety.

### TECHNICAL FIELD

This invention relates to methods and compositions for detecting and assessing functionally active folate receptors on tumors and treatment associated with those tumors. The invention further relates to methods and compositions for selecting ovarian and lung cancer patients for therapy with a folate-vinca conjugate by identifying functionally active folate receptors on the tumors of the patient. The invention also relates to methods and compositions for treating folate receptor expressing epithelial tumors with a folate-vinca conjugate in combination with doxorubicin such as pegylated liposomal doxorubicin in which the tumors include ovarian, endometrial or non-small cell lung cancer tumors, including platinum-resistant ovarian tumors and platinum-sensitive ovarian tumors. The invention also relates to methods and compositions for treating platinum-resistant ovarian cancer using a folate-targeted drug, in the absence or presence of selecting the patient by identifying functionally active folate receptors on the tumors of the patient.

### BACKGROUND AND SUMMARY

An important adjunct to targeted drug therapies is the co-development of diagnostic tests to provide information on the presence or absence of the molecular target in question. For example, selection for therapy with Herceptin® (trastuzumab) is guided by such a diagnostic test, the HercepTest®, a semi-quantitative immunohistochemical (IHC) assay that measures human epidermal growth factor receptor 2 (HER2) expression to aid in selecting patients for treatment with Herceptin®. However, the HercepTest® does not detect functionally active epidermal growth factor receptors (i.e., receptors that bind epidermal growth factor) because antibodies to the epidermal growth factor receptor are used to detect the presence of epidermal growth factor receptors on fixed tissues, not the capacity of those receptors to bind epidermal growth factor.

Following a study with ¹¹¹In-DTPA-folate, to detect folate receptors on the tumors of ovarian cancer patients, studies were initiated to develop a technetium-99m (^{99m}Tc)-based folate linked radiopharmaceutical. Advantages of a technetium-based agent include 1) ready availability of molybdenum/technetium-99m generators, 2) optimal energy (140 keV) for detection in gamma counters, and 3) short half-life. In this regard, ^{99m}Tc-EC20 (EC20) having the formula

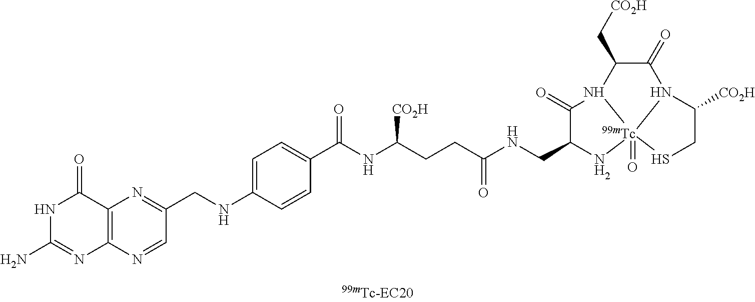

was developed. Technetium-99m-labeled EC20 (^{99m}Tc-EC20) provides real-time, noninvasive detection of tissues expressing folate receptors capable of binding to folate.

The term EC20 is commonly used to identify the non-radioactive reagent lacking a radionuclide:

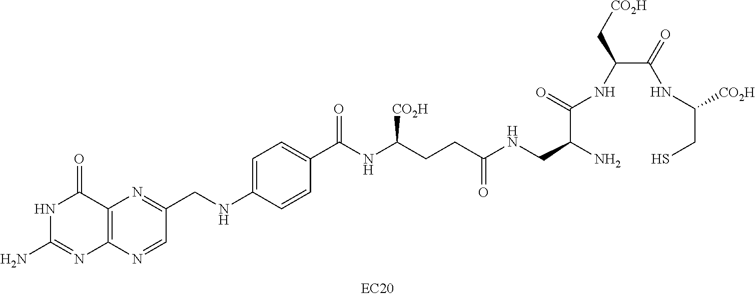

However, EC20 is also commonly used to identify the radioactive drug substance ^{99m}Tc-EC20, which is the substance administered to patients. See Examples 2 and 3, below. In the context of administration to patients for detecting and assessing tissues expressing folate receptors capable of binding to folate, EC20 is used herein to denote the radioactive drug substance ^{99m}Tc-EC20, or a pharmaceutically acceptable salt thereof. It will be appreciated that the substance may be present in solution or suspension in an ionized form, including a deprotonated form.

Folate-targeted drugs have been developed and are being tested in clinical trials as cancer therapeutics. EC145 comprises a highly potent vinca alkaloid cytotoxic compound, desacetylvinblastine hydrazide (DAVLBH), conjugated to folate. The EC145 molecule targets the folate receptor found at high levels on the surface of epithelial tumors, including non-small cell lung carcinomas (NSCLC), ovarian, endometrial and renal cancers, and others, including fallopian tube and primary peritoneal carcinoma. Without being bound by theory, it is believed that EC145 binds to tumors that express the folate receptor delivering the vinca moiety directly to cancer cells while avoiding normal tissue. Upon binding, EC145 enters the cancer cell via endocytosis, releases DAVLBH and causes cell death by inhibiting formation of the mitotic assembly required for cell division. EC145 has the Chemical Abstracts Registry Number 742092-03-1 and the following formula.

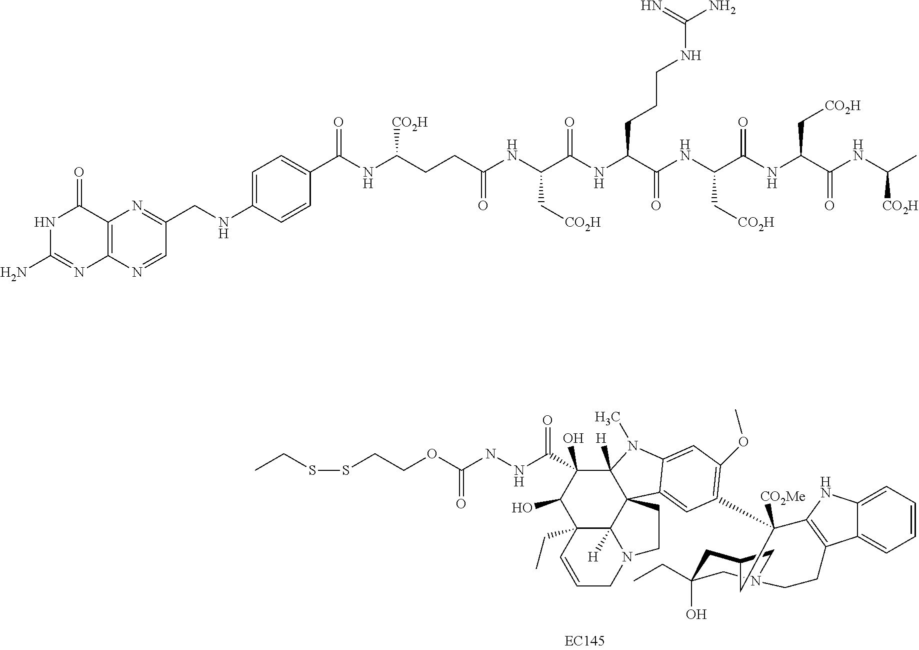

As used herein, in the context of treatment, the term EC145 means the compound, or a pharmaceutically acceptable salt thereof, as indicated above; and the compound may be present in solution or suspension in an ionized form, including a protonated form.

Applicants have demonstrated that folate-radioactive imaging agent conjugates capable of binding to folate receptors, can be used to target a radionuclide to tumors including ovarian tumors or to lung tumors and to further to concentrate the radionuclide in the tumor. Surprisingly, Applicants have discovered that the presence of a threshold level of functionally active folate receptors may be indicative of a clinical benefit to the patient. Thus, in accordance with the invention, a method of determining the presence of active folate receptors on tumors of patients is herein described. In addition, methods for selecting patients for therapy with EC145 are described wherein a patient can be selected for therapy based on a predicted clinical benefit to the patient resulting from detection of a threshold level of functionally active folate receptors on the patient's tumor(s). The clinical benefit to the patient includes progression-free survival of the patient, ability to receive four or more cycles of therapy with EC145, inhibition of tumor growth, stable disease, a partial response of the tumor to therapy, and/or a complete response of the tumor to therapy. Accordingly, the detection of functionally active folate receptors (which may include, but is not limited to, determining a threshold level of expression of functionally active folate receptors) can be used to determine if EC145 is indicated for the treatment of a patient with ovarian cancer or lung cancer. This noninvasive method can be used by medical personnel as an aid in selecting patients for therapy with folate-drug conjugates with ovarian or lung tumors bearing the relevant functionally active folate receptor molecular target.

Applicants have further demonstrated treatment of platinum-resistant ovarian tumors, including metastatic tumors, in patients with a combination of EC145 and pegylated liposomal doxorubicin. Applicants have demonstrated that this combination therapy is advantageous over the treatment of the patients using pegylated liposomal doxorubicin without EC145. EC20 may or may not be used in conjunction with this treatment.

In one aspect of the invention, a method for detecting functionally active folate receptors in patients with tumors is provided.

In another aspect of the invention, there is provided a method for determining the presence of functionally active folate receptors on a tumor such as an ovarian tumor or lung tumor, including primary and metastatic tumors, of a patient comprising the step of administering to the patient a composition comprising EC20.

In another aspect of the invention, there is provided a method of determining whether EC145 is indicated for the treatment of a patient with a tumor such as an ovarian tumor or a lung tumor, the method comprising the step of determining whether functionally active folate receptors are present on the tumor of the patient wherein EC145 is indicated for the treatment of the patient with the tumor if functionally active folate receptors are present on the tumor, including primary and metastatic tumors.

In another aspect, there is provided a method of determining whether EC145 is indicated for the treatment of a patient with an ovarian tumor or a lung tumor, the method comprising the step of administering to the patient EC20, wherein EC145 is indicated for the treatment of the patient with the tumor if the tumor of the patient has functionally active folate receptors wherein the functionally active folate receptors are capable of detection with EC20.

In a further aspect of the invention, there is provided a method of determining whether EC145 is indicated for the treatment of a patient with an ovarian tumor or a lung tumor, the method comprising the step of administering to the patient EC20, wherein EC145 is indicated for the treatment of the patient with the tumor if the radioactive signal produced by the EC20 upon binding to the tumor compared to the background radioactive signal produced by the EC20 is indicative of a clinical benefit to the patient.

In a further aspect of the invention, there is provided a method of predicting a response of an ovarian tumor or a lung tumor of a patient to therapy with EC145, the method comprising the steps of

a) administering to the patient EC20 wherein the EC20 produces a radioactive signal;

b) quantifying the radioactive signal produced by the EC20 upon binding of the EC20 to the tumor;

c) quantifying the background radioactive signal produced by the EC20;

d) comparing the radioactive signal produced upon binding of the EC20 to the tumor to the background radioactive signal; and

e) predicting the response of the tumor to the therapy based on the comparison.

In an additional aspect of the invention, there is provided a method of treatment of folate receptor expressing epithelial tumors in a patient in need thereof comprising administering a therapeutic amount of EC145 in combination with a therapeutic amount of doxorubicin.

In an additional aspect of the invention, there is provided a method of treatment of folate receptor expressing epithelial tumors in a patient in need thereof comprising administering a therapeutic amount of EC145 in combination with a therapeutic amount of pegylated liposomal doxorubicin.

In an additional aspect of the invention, there is provided a method of treatment of platinum-resistant ovarian cancer in a patient in need thereof comprising administering a therapeutic amount of EC145 in combination with a therapeutic amount of pegylated liposomal doxorubicin.

In an additional aspect of the invention, there is provided a method of treatment of platinum-sensitive ovarian cancer in a patient in need thereof comprising administering a therapeutic amount of EC145 in combination with a therapeutic amount of pegylated liposomal doxorubicin.

In a further aspect of the invention, there is provided a method of obtaining a clinical benefit compared to treatment with a therapeutic amount of pegylated liposomal doxorubicin in the treatment of platinum-resistant ovarian cancer in a patient in need thereof comprising administering a therapeutic amount of EC145 in combination with a therapeutic amount of pegylated liposomal doxorubicin

In another aspect, a method of determining whether a patient with a tumor has functionally active folate receptors present on the tumor of the patient is provided. The method comprises the step of administering an effective amount of EC20 to the patient for detection of the functionally active folate receptors. In yet another aspect, the tumor is an ovarian tumor or a lung tumor. In another illustrative aspect, the tumor is a primary tumor or a metastatic tumor. In another embodiment, the functionally active folate receptors are detected visually. In still another aspect, the visual detection of functionally active folate receptors is used to determine folate receptor status of the patient. Illustratively, the folate receptor status of the patient is selected from the group consisting of EC20++, EC20+, and EC20−. In this illustrative aspect, the folate receptor status may be EC20++ and treatment with EC145 is indicated. In another aspect, EC20++ status correlates with a clinical benefit to the patient and the clinical benefit may be disease control rate or overall disease response rate.

### BRIEF DESCRIPTION OF THE DRAWINGS

FIG. 1. Planar Image of a Patient After Administration of ^{99m}Tc-EC20-Folate. Prior to the ^{99m}Tc-EC20 imaging procedure, patients receive one IV injection of 0.5 mg of folic acid, followed, within 1 to 3 minutes, by a 1 to 2 mL injection of 0.1 mg of EC20 labeled with 20 to 25 mCi of technetium-99m. Approximately 1 to 2 hours post-injection of ^{99m}Tc-EC20, mid-thigh to head, anterior and posterior planar images are acquired. Arrows indicate approximate location of the tumors (lesions). In this example, two areas containing folate receptor positive tumors are indicated.

FIG. 2. Planar Image of a Patient After Administration of ^{99m}Tc-EC20-Folate. Prior to the ^{99m}Tc-EC20 imaging procedure, patients receive one IV injection of 0.5 mg of folic acid, followed, within 1 to 3 minutes, by a 1 to 2 mL injection of 0.1 mg of EC20 labeled with 20 to 25 mCi of technetium-99m. Approximately 1 to 2 hours post-injection of ^{99m}Tc-EC20, mid-thigh to head, anterior and posterior planar images are acquired. Arrows indicate approximate location of the tumors (lesions). In this example, two areas containing folate receptor positive tumors are indicated.

FIG. 3. Planar Image of a Patient After Administration of ^{99m}Tc-EC20-Folate. Prior to the ^{99m}Tc-EC20 imaging procedure, patients receive one IV injection of 0.5 mg of folic acid, followed, within 1 to 3 minutes, by a 1 to 2 mL injection of 0.1 mg of EC20 labeled with 20 to 25 mCi of technetium-99m. Approximately 1 to 2 hours post-injection of ^{99m}Tc-EC20, mid-thigh to head, anterior and posterior planar images are acquired. Arrows indicate approximate location of the tumors (lesions). In this example, two areas containing folate receptor positive tumors are indicated.

FIG. 4. Planar Image of a Patient After Administration of ^{99m}Tc-EC20-Folate. Prior to the ^{99m}Tc-EC20 imaging procedure, patients receive one IV injection of 0.5 mg of folic acid, followed, within 1 to 3 minutes, by a 1 to 2 mL injection of 0.1 mg of EC20 labeled with 20 to 25 mCi of technetium-99m. Approximately 1 to 2 hours post-injection of ^{99m}Tc-EC20, mid-thigh to head, anterior and posterior planar images are acquired. Arrows indicate approximate location of the tumors (lesions). In this example, one area containing a folate receptor positive tumor is indicated.

FIG. 5. Planar Image of a Patient After Administration of ^{99m}Tc-EC20-Folate. Prior to the ^{99m}Tc-EC20 imaging procedure, patients receive one IV injection of 0.5 mg of folic acid, followed, within 1 to 3 minutes, by a 1 to 2 mL injection of 0.1 mg of EC20 labeled with 20 to 25 mCi of technetium-99m. Approximately 1 to 2 hours post-injection of ^{99m}Tc-EC20, mid-thigh to head, anterior and posterior planar images are acquired. Arrows indicate approximate location of the tumors (lesions). In this example, six folate receptor positive lesions are indicated.

FIG. 6. CT Scan Image of the Same Patient For Which the Planar Image is Shown in FIG. 5. Regions of interest (high intensity image area within tumor lesion) are indicated by the two ellipses. Images were measured prior to commencement of treatment with EC145 to yield the following sizes: Tumor 1-34 mm, Tumor 2-25 mm.

FIG. 7. CT Scan Image of the Same Patient For Which the Planar Image is Shown in FIG. 5. Regions of interest (high intensity image area within tumor lesion) are indicated by the two ellipses. Images were measured after 8 weeks (2 cycles) of treatment with EC145 to yield the following tumor sizes (percent size change): Tumor 1-15 mm (−56%), Tumor 2-10 mm (60%).

FIG. 8. Exemplary 16 week treatment regimen with EC145.

FIG. 9. Tumor Response of Non-Small Cell Lung Carcinoma and Ovarian Cancer Tumors to Treatment. Tumors were divided into two groups, folate-receptor positive and folate-receptor negative (separated by the vertical dotted line in the figure), based on imaging results after administration of ^{99m}Tc-EC20 according to the methods described in Example 16. The change in size of each tumor after treatment by the method of Example 18 or Example 19 is indicated by the individual bars in the graph. As described in Example 21, the mean increase in size for all tumors that were folate-receptor positive based on the method described in Example 16 was significantly less than the mean increase in size for all tumors that were folate-receptor negative, 7% versus 33%, respectively.

FIG. 10. SPECT and planar images showing EC20 uptake in target lesions. ^{99m}Tc-EC20 allows the physician to obtain a real-time assessment of receptor expression. Panels A, B, and C compare CT, SPECT and planar images from an ovarian cancer patient (patient 035, study EC-FV-02) showing ^{99m}Tc-EC20 uptake in abdominal masses (white arrows). Panel A—CT scan; Panel B—SPECT image showing ^{99m}Tc-EC20 uptake; Panel B-planar image showing ^{99m}Tc-EC20 uptake.

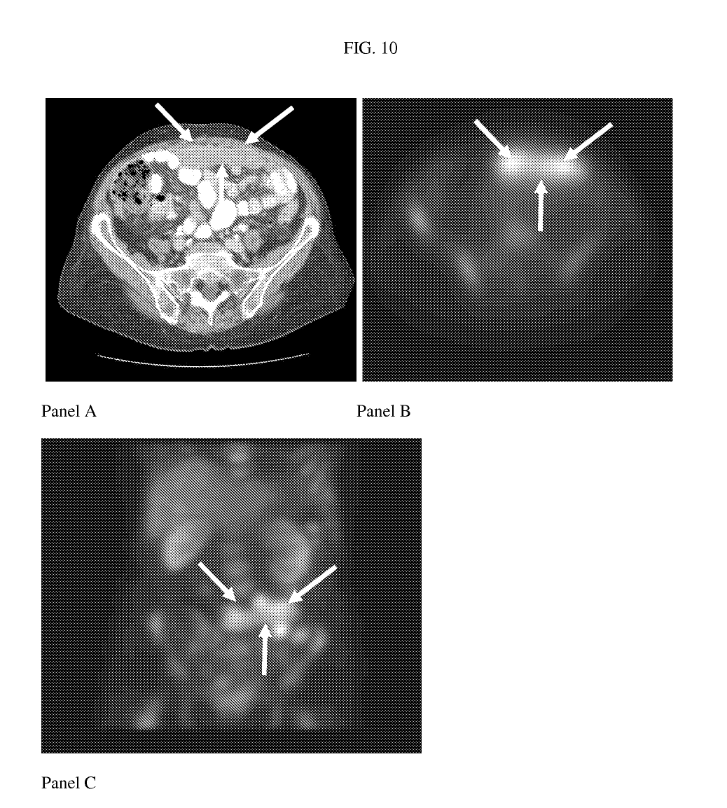

FIG. 11 shows the Kaplan-Meier curves for progression free survival (PFS) at the interim analysis in study EC-FV-04 for patients treated with EC145 in combination with pegylated liposomal doxorubicin (EC145+PLD) and for patients treated with pegylated liposomal doxorubicin alone (PLD alone).

FIG. 12 shows Kaplan-Meier curves for progression free survival (PFS) time in Study EC-FV-04, an ongoing phase 2 trial in women with platinum-resistant ovarian cancer, at the time of the interim analysis, for subjects enrolled at sites with nuclear imaging capabilities who were scanned with EC20 prior to study treatment and assessed as EC20 positive (EC20++ status) prior to study treatment (EC145 in combination with PLD versus PLD alone).

FIG. 13 shows Kaplan-Meier curves for overall survival (OS) time in Study EC-FV-02, a trial in women with advanced ovarian and endometrial cancers who were scanned with EC20 prior to study treatment and assessed as EC20 positive (EC20++ status) compared to those assessed as EC20+ status or EC20− status prior to study treatment. This curve shows the utility of selecting patients who benefit from the single agent EC145 in highly refractory ovarian cancer patients.

FIG. 14 shows Kaplan-Meier curves for overall survival (OS) time in Study EC-FV-04, an ongoing phase 2 trial in women with platinum-resistant ovarian cancer, at the time of the interim analysis, for patients treated with EC145 in combination with pegylated liposomal doxorubicin (EC145+PLD) and for patients treated with pegylated liposomal doxorubicin alone (PLD alone).

FIG. 15 shows the synergistic relationship between EC145 and doxorubicin in the inhibition of growth of KB tumor cells in vivo as described in Example 7; data points that fall below the line represent synergism.

FIG. 16 shows the effects on tumor growth and responses (PR=partial response, CR=complete response, Cures) from the study in mice bearing M109 tumors described in Example 8 for the following groups: (a) M109 control; (b) EC145, 2 μmol/kg; (c) DOXIL, 7 mg/kg; (d) EC145, 2 μmol/kg+DOXIL, 7 mg/kg; (e) DOXIL, 4 mg/kg; and (f) EC145, 2 μmol/kg+DOXIL, 4 mg/kg.

FIG. 17 shows the effects on weight change from the study in mice bearing M109 tumors described in Example 8 for the following groups: (a) M109 control; (b) EC145, 2 μmol/kg; (c) DOXIL, 7 mg/kg; (d) EC145, 2 μmol/kg+DOXIL, 7 mg/kg; (e) DOXIL, 4 mg/kg; and (f) EC145, 2 μmol/kg+DOXIL, 4 mg/kg.

### DEFINITIONS

In accordance with the invention, “functionally active folate receptors” means folate receptors expressed on an ovarian or a lung tumor at a tumor to background ratio of at least about 1.2 or greater. The term also can be used to mean a signal from tumors detectable visually (e.g., used to identify an EC20++ patient as described below). The presence of “functionally active folate receptors” (i.e., a tumor to background ratio of at least about 1.2 or greater or a signal from tumors detected visually) correlates with a clinical benefit to a patient selected for therapy with EC145, the clinical benefit including progression-free survival of the patient, overall survival of the patient, ability to receive four or more cycles of therapy with EC145, inhibition of tumor growth, stable disease, a partial response, and/or a complete response.

In accordance with the invention, “tumor to background ratio” means the ratio of the radioactive signal produced by EC20 upon binding to a tumor compared to the background radioactive signal produced by the folate-radioactive imaging agent in the patient.

In accordance with the invention, “clinical benefit” means a response of a patient to treatment with EC145 where the response includes progression-free survival of the patient, overall survival of the patient, ability to receive four or more cycles of therapy (e.g., four weeks of therapy) with EC145, inhibition of tumor growth, stable disease, a partial response, and/or a complete response.

In accordance with the invention, “inhibition of tumor growth” means reduction in tumor size, complete disappearance of a tumor, or growth of a patient tumor of less than 30% over the course of therapy with EC145.

In accordance with the invention, “stable disease” means no material progression of disease in a patient over the course of therapy with EC145.

In accordance with the invention, “a partial response” means a decrease in tumor size of 30% or greater in a patient treated with EC145.

In accordance with the invention, “a complete response” means the disappearance of detectable disease in a patient treated with EC145.

### DETAILED DESCRIPTION OF ILLUSTRATIVE EMBODIMENTS

In any of the various disclosures above, the following features may be present where applicable, providing additional embodiments of the invention.

Another embodiment is described wherein the method further comprises the step of administering to the patient an unlabeled folate, such as folic acid or a salt thereof, prior to administration of EC20, in the form of a complex with a radionuclide.

Another embodiment is described wherein EC145 is indicated for the treatment of the patient with the tumor if the radioactive signal produced by EC20 upon binding to the tumor compared to the background radioactive signal produced by the EC20 is indicative of a clinical benefit to the patient.

Another embodiment is described wherein the clinical benefit is progression-free survival of the patient.

Another embodiment is described wherein the clinical benefit is inhibition of tumor growth.

Another embodiment is described wherein the clinical benefit is selected from the group consisting stable disease, a partial response, and a complete response.

Another embodiment is described wherein the level of expression of the functionally active folate receptors is quantified based on a tumor to background ratio of the radioactive signal produced by the EC20 to the background radioactive signal.

Another embodiment is described wherein the tumor to background ratio is at least about 1.2.

Another embodiment is described wherein the tumor to background ratio is at least about 1.3.

Another embodiment is described wherein the tumor to background ratio is at least about 1.4.

Another embodiment is described wherein the tumor is an ovarian tumor.

Another embodiment is described wherein the tumor is a platinum-resistant ovarian tumor.

Another embodiment is described wherein the tumor is a lung tumor.

Another embodiment is described wherein the tumor is a non-small cell carcinoma of the lung.

Another embodiment is described wherein either the EC145, the EC20, or both are in a parenteral dosage form.

Another embodiment is described wherein the dosage form is selected from the group consisting of intradermal, subcutaneous, intramuscular, intraperitoneal, intravenous, and intrathecal.

Another embodiment is described wherein the EC145 is in a composition and wherein the composition further comprises a pharmaceutically acceptable carrier.

Another embodiment is described wherein the composition comprising the EC20 further comprises a pharmaceutically acceptable carrier.

Another embodiment is described wherein the pharmaceutically acceptable carrier is a liquid carrier.

Another embodiment is described wherein the liquid carrier is selected from the group consisting of saline, glucose, alcohols, glycols, esters, amides, and a combination thereof.

Another embodiment is described wherein the EC145 is administered in a therapeutically effective amount.

Another embodiment is described wherein the EC20 is administered in a therapeutically effective amount. For EC20, a therapeutically effective amount denotes a diagnostically effective amount.

Another embodiment is described wherein the effective amount ranges from about 1 ng to about 1 mg per kilogram of body weight.

Another embodiment is described wherein the effective amount ranges from about 100 ng to about 500 μg per kilogram of body weight.

Another embodiment is described wherein the effective amount ranges from about 100 ng to about 50 μg per kilogram of body weight.

Another embodiment is described wherein the tumor is a primary tumor.

Another embodiment is described wherein the tumor is a metastasized tumor.

Another embodiment is described wherein the EC20 is radiolabeled using a chelating agent and a reducing agent.

Another embodiment is described wherein the chelating agent is sodium α-D-glucoheptonate.

Another embodiment is described wherein the reducing agent is tin (II) chloride dihydrate.

Another embodiment is described further comprising the step of administering to the patient doxorubicin. One embodiment is wherein the doxorubicin is in the form of a pegylated liposomal doxorubicin (PLD).

For any method or use described herein for EC20, or a pharmaceutically acceptable salt thereof, an alternative embodiment is a folate-radioactive imaging conjugate having as the complexed radionuclide a cation of a radionuclide selected from the group consisting of isotopes of gallium, indium, copper, technetium, and rhenium.

For all of the embodiments, any applicable combination of embodiments is also contemplated. Any applicable combination of the above-described embodiments is considered to be in accordance with the invention.

In accordance with the invention, EC20 can be used to target a radionuclide to ovarian tumors or to lung tumors and further to concentrate the radionuclide in the tumor for use in detecting functionally active folate receptors on the tumors. Surprisingly, Applicants have discovered that a threshold level of folate receptor expression on the tumor (i.e., the presence of functionally active folate receptors on the tumor) correlates with a clinical benefit to a patient selected for therapy with EC145. Thus, in accordance with the invention, a method of determining the presence of functionally active folate receptors on tumors of patients is herein described. In addition, methods are provided for selecting patients for therapy with EC145 wherein a patient can be selected for therapy based on a predicted clinical benefit resulting from detection of a threshold level of functionally active folate receptors on the patient's tumor. The clinical benefit to the patient includes progression-free survival of the patient, overall survival of the patient, ability to receive four or more cycles of therapy with EC145, inhibition of tumor growth, stable disease, a partial response of the tumor to therapy, and/or a complete response of the tumor to therapy. The threshold level of folate receptor expression can be, for example, a tumor to background ratio of at least about 1.2, at least about 1.3, or at least about 1.4, or can be detected visually (e.g., visual detection used to identify an EC20++ patient as described below). Accordingly, the detection of functionally active folate receptors (i.e., a threshold level of folate receptor expression detected as a tumor background ratio or detected visually, for example) can be used to determine if EC145 is indicated for the treatment of a patient with an ovarian tumor or a lung tumor.

In one embodiment, the method is applicable to tumor types having functionally active folate receptors including ovarian tumors or lung tumors. In another illustrative embodiment, the method is applicable to platinum-resistant ovarian tumors. In yet another embodiment, the method is applicable to non-small cell lung carcinomas. In another illustrative embodiment, the tumor can be a primary tumor. In another embodiment, the tumor can be a metastasized tumor.

In one embodiment, the method described herein is used to quantify functionally active folate receptors.

In another embodiment, the method described herein is used to quantify functionally active folate receptors to determine if EC145 is indicated for the treatment of a patient with an ovarian tumor or a lung tumor. In one embodiment the patient, optionally, can be preinjected with unlabeled folate and then injected with ^{99m}Tc-EC20 to determine a tumor to background ratio. In this embodiment, a tumor to background ratio is the ratio of the radioactive signal (e.g., by SPECT/CT or SPECT imaging) produced by ^{99m}Tc-EC20 upon binding to the tumor compared to the background radioactive signal produced by the folate-radioactive imaging agent in the patient. In this embodiment the tumor to background ratio can be, for example, at least about 1.2. Alternatively, the presence of a threshold level of functionally active folate receptors can be determined visually, e.g., to identify an EC20++ patient as described below.

The threshold level of expression of functionally active folate receptors may correlate with a clinical benefit to the patient. The clinical benefit can include progression free survival of the patient, overall survival of the patient, ability to receive four or more cycles of therapy with EC145, inhibition of tumor growth, stable disease, a partial response of the tumor to therapy, and/or a complete response of the tumor to therapy. The detection of functionally active folate receptors (e.g., a threshold level of folate receptor expression reflected in a tumor to background ratio of 1.2 or determined visually, e.g., visual detection used to identify an EC20++ patient as described below) can be used to determine if EC145 is indicated for the treatment of a patient with an ovarian tumor or a lung tumor.

In the above described embodiment the tumor to background ratio can be, for example, 1.2, 1.3 or 1.4, or detected visually. In another illustrative embodiment the threshold level of functionally active folate receptors can be determined by visual examination of, for example, a predetermined region of a SPECT/CT or SPECT image and coding the intensity of ^{99m}Tc-EC20 uptake as, for example, no uptake, mild uptake, or marked uptake, and selecting patients for therapy with mild uptake or marked uptake.

In yet another embodiment, a method of selecting a patient with an ovarian tumor or a lung tumor for therapy with a conjugate comprising a folate linked to a vinca compound is described. The method comprises the step of determining if functionally active folate receptors are present on the tumor of the patient wherein the patient is selected for therapy with the folate-vinca compound conjugate if functionally active folate receptors are detected on the tumor.

In another embodiment, a method of selecting a patient with an ovarian tumor or a lung tumor for therapy with a conjugate comprising a folate linked to a vinca compound is described. The method comprises the step of administering to the patient a composition comprising a folate linked to a radioactive imaging agent, wherein the patient is selected for the therapy with the conjugate comprising the folate linked to the vinca compound if the tumor of the patient has functionally active folate receptors wherein the functionally active folate receptors are capable of detection with the EC20.

In another embodiment, a method of selecting a patient with an ovarian tumor or a lung tumor for therapy with a conjugate comprising a folate linked to a vinca compound is described. The method comprises the step of administering to the patient a conjugate comprising a folate linked to a radioactive imaging agent, wherein the patient is selected for therapy if the radioactive signal produced by the EC20 upon binding to the tumor compared to the background radioactive signal produced by the EC20 is indicative of a clinical benefit to the patient.

In one embodiment of the invention, the EC20 can be administered to the patient in combination with unlabeled folate. “In combination with” means that the unlabeled vitamin can be either coadministered with the EC20 or the unlabeled folate can be preinjected before administration of the EC20 to improve image quality. For example, the EC20 can be administered in combination with about 0.5 ng unlabeled folate/kg of body weight to about 100 mg unlabeled folate/kg of body weight, or about 1 μg unlabeled folate/kg of body weight to about 100 mg unlabeled folate/kg of body weight, or about 100 μg unlabeled folate/kg of body weight to about 100 mg unlabeled folate/kg of body weight, or about 100 μg unlabeled folate/kg of body weight to about 700 μg unlabeled folate/kg of body weight, with an average patient having a body weight of about 70 kg.

Another embodiment is a method of determining whether a patient with a tumor has functionally active folate receptors present on the tumor of the patient. In one embodiment the tumor is an ovarian tumor or a lung tumor. In another embodiment the tumor is a primary tumor or a metastatic tumor. In a further embodiment the method comprises administering to a patient an effective amount of Tc-EC20 for detection of the functionally active folate receptors.

In other embodiments of the methods described herein, pharmaceutically acceptable salts of the conjugates described herein are described. Pharmaceutically acceptable salts of the conjugates described herein include the acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In various embodiments of the methods described herein, the EC145 may be administered alone or in combination with one or more other drugs (or as any combination thereof). In one illustrative embodiment, the EC145 can be administered in combination with doxorubicin. In one illustrative embodiment, the EC145 is administered in combination with pegylated liposomal doxorubicin as described in Example 20.

In one embodiment, the conjugates described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated to be for immediate and/or modified release. In one illustrative aspect, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates described herein or compositions comprising the conjugates may be continuously administered, where appropriate.

In one embodiment, a kit is provided. If a combination of active compounds is to be administered, two or more pharmaceutical compositions may be combined in the form of a kit suitable for sequential administration or co-administration of the compositions. Such a kit comprises two or more separate pharmaceutical compositions, at least one of which contains a conjugate described herein, and means for separately retaining the compositions, such as a container, divided bottle, or divided foil packet. In another embodiment, compositions comprising one or more conjugates described herein, in containers having labels that provide instructions for use of the conjugates for patient selection and/or treatment are provided.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active compound into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering the EC145 can be used. For example, the EC145 can be administered as single doses, or can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of the EC145 to eliminate the tumor. In one embodiment, the patient is injected multiple times (preferably about 2 up to about 50 times) with the EC145, for example, at 12-72 hour intervals or at 48-72 hour intervals. Additional injections of the EC145 can be administered to the patient at an interval of days or months after the initial injections(s) and the additional injections can prevent recurrence of the cancer.

Any suitable course of therapy with the EC145 can be used. In one embodiment, individual doses and dosage regimens are selected to provide a total dose administered during a month of about 15 mg. In one illustrative example, the EC145 is administered in a single daily dose administered five days a week, in weeks 1, 2, and 3 of each 4 week cycle, with no dose administered in week 4. In an alternative example, the EC145 is administered in a single daily dose administered three days a week, of weeks 1, and 3 of each 4 week cycle, with no dose administered in weeks 2 and 4.

The unitary daily dosage of the EC145 can vary significantly depending on the patient condition, the disease state being treated, the molecular weight of the EC145, its route of administration and tissue distribution, and the possibility of co-usage of other therapeutic treatments, such as radiation therapy or additional drugs in combination therapies. The effective amount to be administered to a patient is based on body surface area, mass, and physician assessment of patient condition. Effective doses can range, for example, from about 1 ng/kg to about 1 mg/kg, from about 1 μg/kg to about 500 μg/kg, and from about 1 μg/kg to about 100 μg/kg. These doses are based on an average patient weight of about 70 kg.

The conjugates described herein can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg. The physician will readily be able to determine doses for subjects whose weight falls outside this range, such as infants and the elderly.

The conjugates described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The conjugates described herein may be capable of existing as geometric isomers. Accordingly, it is to be understood that the present invention includes pure geometric isomers or mixtures of geometric isomers.

It is appreciated that the conjugates described herein may exist in unsolvated forms as well as solvated forms, including hydrated forms. In general, the solvated forms are equivalent to unsolvated forms and are encompassed within the scope of the present invention. The conjugates described herein may exist in multiple crystalline or amorphous forms. In general, all physical forms are equivalent for the uses contemplated by the present invention and are intended to be within the scope of the present invention.

In another embodiment, compositions and/or dosage forms for administration of EC145 are prepared from EC145 with a purity of at least about 90%, or about 95%, or about 96%, or about 97%, or about 98%, or about 99%, or about 99.5%. In another embodiment, compositions and or dosage forms for administration of EC145 are prepared from EC145 with a purity of at least 90%, or 95%, or 96%, or 97%, or 98%, or 99%, or 99.5%.

In another embodiment, compositions and/or dosage forms for administration of EC20 are prepared from EC20 with a purity of at least about 90%, or about 95%, or about 96%, or about 97%, or about 98%, or about 99%, or about 99.5%. In another embodiment, compositions and or dosage forms for administration of EC20 are prepared from EC20 with a purity of at least 90%, or 95%, or 97%, or 98%, or 99%, or 99.5%.

In another embodiment, compositions and/or dosage forms for administration of radiolabeled EC20 are prepared from EC20 of with a radiochemical purity of at least about 90%, or about 95%, or about 96%, or about 97%, or about 98%, or about 99%, or about 99.5%. In another embodiment, compositions and or dosage forms for administration of EC20 are prepared from EC20 with a purity of at least 90%, or 95%, or 96%, or 97%, or 98%, or 99%, or 99.5%.

As used herein, purity determinations may be based on weight percentage, mole percentage, and the like. In addition, purity determinations may be based on the absence or substantial absence of certain predetermined components, such as, but not limited to, folic acid, disulfide containing components not containing a vinca drug, oxidation products, disulfide components not containing a folate, and the like. It is also to be understood that purity determinations are applicable to solutions of the compounds and compositions purified by the methods described herein. In those instances, purity measurements, including weight percentage and mole percentage measurements, are related to the components of the solution exclusive of the solvent.

The purity of the EC145 or the EC20 may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In one aspect, patient response to treatment was characterized utilizing Response Evaluation Criteria in Solid Tumors (RECIST) criteria. Illustratively, the criteria have been adapted from the original WHO Handbook (3), taking into account the measurement of the longest diameter for all target lesions: complete response, (CR)—the disappearance of all target lesions; partial response (PR)—at least a 30% decrease in the sum of the longest diameter of target lesions, taking as reference the baseline sum longest diameter; stable disease (SD)—neither sufficient shrinkage to qualify for partial response nor sufficient increase to qualify for progressive disease, taking as reference the smallest sum longest diameter since the treatment started; progressive disease (PD)—at least a 20% increase in the sum of the longest diameter of target lesions, taking as reference the smallest sum longest diameter recorded since the treatment started or the appearance of one or more new lesions. Overall disease response rate (ORR) is calculated as the percent of patients who achieve a best response of CR or PR. Overall disease control rate (DCR) is calculated as the percent of patients who achieve a best response of CR, PR, or SD.

In another embodiment, the EC145 is provided in a sterile container or package. In another embodiment, EC20 is provided in a sterile container or package.

In one embodiment, a method is provided of determining whether EC145 is indicated for the treatment of a patient with one or more ovarian tumors or one or more lung tumors, the method comprising the step of determining a folate-receptor status in a patient with ovarian cancer wherein the EC145 is indicated for the treatment of the patient if the folate-receptor status in the patient is positive.

As used herein, when used in patients, the term “EC20” refers to EC20, or pteroyl-γ-D-glutamyl-β-L-2,3-diaminopropionyl-L-aspartyl-L-cysteine or pteroyl-γ-D-glutamyl-β-L-2,3-diaminopropionyl-L-aspartyl-L-cysteine complexed to ^{99m}Tc; for example, the term “^{99m}Tc-EC20” explicitly refers to the complex containing the radioactive ^{99m}Tc.

Folate-receptor status in the patient is positive if one or more tumors in the patient have folate receptors capable of binding EC20 or if all tumors in the patient are capable of binding EC20. In one illustrative example, the folate-radioactive imaging agent conjugate is ^{99m}Tc-EC20. At the time of the interim analysis described in Example 25, below, 91.3% of all ovarian cancer patients scanned with EC20 had been “positive” (indicated by having at least one tumor lesion/area that binds EC20) versus 8.7% of patients that were fully EC20 “negative”.

In one embodiment, a method is provided of assessing whether EC145 is indicated for the treatment of a patient with one or more ovarian tumors or one or more lung tumors. The method comprises the steps of visually determining folate receptor status (e.g., EC20++, EC20+, or EC20−) in the patient wherein folate receptor status is based on a measurement of the percentage of evaluated tumors that are folate receptor positive in the patient, and wherein the EC145 is indicated for the treatment of the patient when the folate receptor status of the patient is EC20++. In an illustrative embodiment, EC20++ status means that the percentage of evaluated tumors in the patient that are folate receptor positive is about 100%. In other illustrative aspects, EC20++ status means that the percentage of evaluated tumors in the patient that are folate receptor positive is about 90%, about 80%, or about 70%. In another aspect, EC20 is a semi-quantitative imaging agent.

In this visual assessment embodiment (visual detection), lesions are evaluated visually to determine if the patient has a threshold level of functionally active folate receptors indicative of a clinical benefit to the patient. In one aspect, lesions (i.e., tumors) for analysis in each patient are selected by a radiologist according to RECIST (v1.0) criteria. Subsequently, a nuclear medicine physician (i.e. reader) assesses the uptake of the EC20 for each evaluable target lesion visually, and classifies the uptake as “EC20 positive” (marked uptake/mild uptake) or “EC20 negative” (no uptake). In one illustrative example the folate-radioactive imaging agent conjugate is ^{99m}Tc-EC20. The term “no uptake” means that visual inspection of the target lesion compared with the nearby tissue indicates that uptake of EC20 in the target lesion and uptake of EC20 in nearby tissue are not distinguishable. The term “mild uptake” means that visual inspection of the target lesion compared with the nearby tissue indicates that uptake of EC20 in the target lesion and uptake of EC20 in nearby tissue are distinguishable. The term “marked uptake” means that visual inspection of the target lesion compared with the nearby tissue indicates that uptake of EC20 in the target lesion and uptake of EC20 in nearby tissue are clearly distinguishable.

In this embodiment, lesions can be evaluable or non-evaluable. In one embodiment, lesions less than 1.5 cm in longest dimension (LD) are considered “non-evaluable” unless the nuclear medicine reader identified them as having unequivocal uptake of EC20, in which case they are characterized as “positive.” Moreover, certain organs (e.g., liver, spleen, bladder, and kidney) have an inherently high uptake of EC20. Target lesions located in these organs are considered “non-evaluable.”

In another embodiment, EC20 non-evaluable lesions fit one of the following criteria: 1) defined as “not imaged” or “not applicable” on 99mTc-EC20 SPECT target lesion evaluation 2) as negative for EC20 uptake and less than 15 mm in diameter or 3) lesion located in the liver, kidney/adrenal gland, spleen, or bladder. EC20 evaluable lesions fit one of the following criteria: 1) defined as positive for EC20 uptake, 2) defined as negative for EC20 uptake and greater than or equal to 15 mm in diameter.

In one embodiment, patients are assigned to groups (i.e. assigned a status) based on the observation of EC20 positive lesions, EC20 negative lesions, and/or non-evaluable lesions in the patient. The percentage of lesions that are EC20 positive in each patient is calculated as follows: % EC20 positive lesions=(number of EC20 positive lesions/number of EC20 negative lesions+number of non-evaluable lesions). In one illustrative example, patients are assigned to three groups denoted EC20++, EC20+, and EC20− wherein about 100% of the lesions in the patients assigned to the EC20++ group are EC20 positive; from about 1% to about 99% of the lesions in the patients assigned to the EC20+ group are EC20 positive; and about 0% of the lesions in the patients assigned to the EC20− group are EC20 positive. In another illustrative example, patients are assigned to three groups denoted EC20++, EC20+, and EC20− wherein about 90% of the lesions in the patients assigned to the EC20++ group are EC20 positive; from about 11% to about 89% of the lesions in the patients assigned to the EC20+ group are EC20 positive; and about 0 to about 10% of the lesions in the patients assigned to the EC20− group are EC20 positive.

In the above-described embodiment, if a patient is in the EC20++ group, a clinical benefit of EC145 treatment is indicated. The clinical benefit to the patient includes progression-free survival of the patient, overall survival of the patient, ability to receive four or more cycles of therapy with EC145, inhibition of tumor growth, stable disease, a partial response of the patient to therapy, a complete response of the patient to therapy, disease control (i.e., the best result obtained is a complete response, a partial response, or stable disease), and/or overall disease response (i.e., the best result obtained is a complete response or a partial response). In one illustrative example, the clinical benefit for a patient being treated for non-small cell lung cancer is determined at 4 months after the beginning of the treatment. In another illustrative example, the clinical benefit for a patient being treated for ovarian cancer is determined at 6 months after the beginning of the treatment.

In one illustrative example overall survival is the time to death for a given patient defined as the number of days from the first day the patient received protocol treatment (C1D1) to the date of the patient's death. All events of death can be included, regardless of whether the event occurred while the patient was still taking the study drug or after the patient discontinued the study drug. If a patient has not died, then the data can be censored at the last study visit, or the last contact date, or the date the patient was last known to be alive, whichever is last.

In an in vitro study described below in Example 7, EB 145 and doxorubicin have been shown to synergistically inhibit the growth of human cancer KB tumor cells.

In a study in mice bearing the Madison 109 lung carcinoma (M109), a folate receptor (FR)-(over)expres sing epithelial tumor relatively resistant to chemotherapy described below in Example 8, it has been demonstrated that EC145 in combination with pegylated liposomal doxorubicin (PLD), trade names Doxil® and Caelyx®, displayed an excellent anti-tumor effect and cure rate, with mild weight loss. Accordingly, in one embodiment there is provided a method of treatment of a folate receptor expressing epithelial tumor in a patient in need thereof comprising administering a therapeutic amount of EC145 in combination with a therapeutic amount of doxorubicin. Another embodiment is the use of EC145 in combination with doxorubicin for the treatment of a folate receptor expressing epithelial tumor in a patient. A further embodiment is the use of EC145 for the manufacture of a medicament for the treatment in combination with doxorubicin of a folate receptor expressing epithelial tumor in a patient.

A further embodiment is a method of achieving a clinical benefit in the treatment of a folate receptor expressing epithelial tumor in a patient in need thereof comprising administering a therapeutic amount of EC145 in combination with a therapeutic amount of doxorubicin. In one embodiment, the clinical benefit is progression-free survival. In another embodiment, the clinical benefit is overall survival.

For any of the above methods or uses, in one embodiment, the doxorubicin is in the form of a pegylated liposomal doxorubicin.

For any of the above methods or uses, an embodiment of a folate receptor expressing epithelial tumor is an ovarian, endometrial or non-small cell lung cancer (NSCLC) tumor. For any of the above methods or uses, another embodiment of a folate receptor expressing epithelial tumor is an ovarian tumor.

Ovarian cancer patients who respond to initial platinum-containing systemic therapy, only to experience disease progression after a treatment-free interval of less than 6 months, are considered, by convention, to have platinum-resistant disease. These patients are considered to have failed primary platinum therapy. An additional group of patients may respond to initial platinum-containing systemic therapy and progress longer than 6 months after therapy. These patients may receive additional platinum-containing therapy, only to progress while on, or within 6 months of having received, secondary platinum therapy. Also deemed platinum resistant, these patients are considered to have failed secondary platinum therapy.

Patients with platinum-resistant disease have a limited number of therapeutic options, and often receive agents such as topotecan, gemcitabine, and pegylated liposomal doxorubicin (PLD); the latter approved in the United States under the trade name Doxil® and elsewhere under the trade name Caelyx®, for the treatment of patients with ovarian cancer whose disease has progressed or recurred after platinum-based chemotherapy. Indeed, PLD is frequently used as treatment for patients with recurrent platinum-resistant ovarian cancer. PLD is a polyethylene glycol liposomal encapsulation of doxorubicin, an anthracycline topoisomerase inhibitor, known to have broad antitumor activity. The liposomal encapsulation provides altered pharmacokinetics over the parent compound, including a prolonged circulation half-life (see Doxil® Package Insert).

In one embodiment there is provided a method of treatment of platinum-resistant ovarian cancer in a patient in need thereof comprising administering a therapeutic amount of EC145 in combination with a therapeutic amount of pegylated liposomal doxorubicin. Another embodiment is the use of EC145 in combination with pegylated liposomal doxorubicin for the treatment of platinum-resistant ovarian cancer in a patient. A further embodiment is the use of EC145 for the manufacture of a medicament for the treatment in combination with pegylated liposomal doxorubicin of platinum-resistant ovarian cancer in a patient.

A further embodiment is a method of achieving a clinical benefit in the treatment of platinum-resistant ovarian cancer in a patient in need thereof comprising administering a therapeutic amount of EC145 in combination with a therapeutic amount of pegylated liposomal doxorubicin. In one embodiment, the clinical benefit is progression-free survival. In another embodiment, the clinical benefit is overall survival.

In a further embodiment of the invention, there is provided a method of treatment of platinum sensitive ovarian cancer in a patient in need thereof comprising administering a therapeutic amount of EC145 in combination with a therapeutic amount of pegylated liposomal doxorubicin or doxorubicin which is not of the pegylated liposomal form. A further embodiment is the use of EC145 for the manufacture of a medicament for the treatment in combination with pegylated liposomal doxorubicin or doxorubicin which is not of the pegylated liposomal form of platinum-sensitive ovarian cancer in a patient.

A further embodiment is a kit comprising a therapeutic amount of EC145 and a therapeutic amount of pegylated liposomal doxorubicin in separate containers.

In another embodiment for any method, use or kit, the EC145 is a compound having the formula

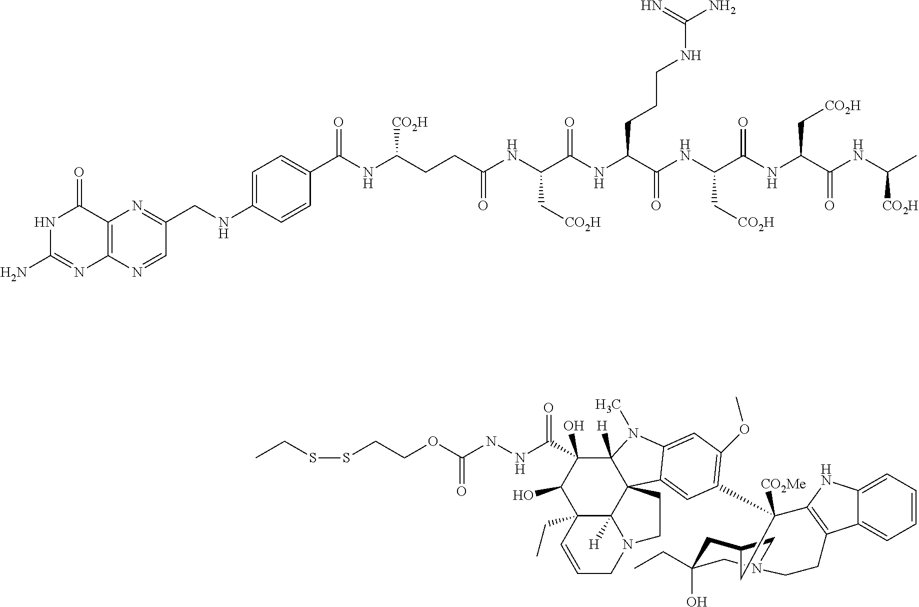

or a pharmaceutically acceptable salt thereof.

As used herein, EC145 may be present in solution or suspension in an ionized form, including a protonated form.

In one embodiment, there is provided a method of treatment of platinum-resistant ovarian cancer in a patient in need thereof comprising administering a therapeutic amount of EC145 in combination with a therapeutic amount of pegylated liposomal doxorubicin. In another embodiment, there is provided use of EC145 in combination with pegylated liposomal doxorubicin for the treatment of platinum-resistant ovarian cancer in a patient. In another embodiment, there is provided the use of EC145 for the manufacture of a medicament for the treatment in combination with pegylated liposomal doxorubicin of platinum-resistant ovarian cancer in a patient.

In a further embodiment, there is provided a method of obtaining a clinical benefit compared to treatment with a therapeutic amount of pegylated liposomal doxorubicin in the treatment of platinum-resistant ovarian cancer in a patient in need thereof, comprising administering a therapeutic amount of EC145 in combination with a therapeutic amount of pegylated liposomal doxorubicin. In one embodiment, the clinical benefit is progression-free survival. In another embodiment, the clinical benefit is overall survival.

The utility of EC145 in combination with pegylated liposomal doxorubicin in treatment of platinum-resistant ovarian cancer is demonstrated in the clinical trial results provided in the Examples below, as well as in the figures.

For any method or use described above concerning the treatment of platinum-resistant ovarian cancer using EC145 in combination with pegylated liposomal doxorubicin, one embodiment is one wherein the purity of EC145 is at least 90%. Another embodiment is one wherein the EC145 is provided in an aqueous sterile liquid formulation the components of which comprise monobasic sodium phosphate monohydrate, dibasic disodium phosphate dihydrate, sodium chloride, potassium chloride and water for injection.

A further embodiment is one wherein the treatment further comprises a bowel regimen. A suggested progressive bowel regimen can be modified from Carney Mont., Meier Del. Palliative care and end-of-life issues. Anaesthesiol Clin North America 2000;18:183.

In one embodiment, the bowel regimen comprises administering Docusate, 100 mg twice daily (b.i.d.) and Senna, 1 tablet once daily (q.d.) or b.i.d.

In one embodiment, the bowel regimen comprises administering Docusate, 100 mg b.i.d., Senna, 2 tablets b.i.d., and Bisacodyl rectal suppositories, 1-2 after breakfast.

In one embodiment, the bowel regimen comprises administering Docusate, 100 mg b.i.d., Senna, 3 tablets b.i.d., and Bisacodyl rectal suppositories, 3-4 after breakfast.

In one embodiment, the bowel regimen comprises administering Docusate, 100 mg b.i.d., Senna, 4 tablets b.i.d., Lactulose or sorbitol, 15 mL b.i.d., and Bisacodyl rectal suppositories, 3-4 after breakfast.

In one embodiment, the bowel regimen comprises administering Docusate, 100 mg b.i.d., Senna, 4 tablets b.i.d., Lactulose or sorbitol, 30 ml b.i.d., and Bisacodyl rectal suppositories, 3-4 after breakfast.

In one embodiment, the bowel regimen comprises administering Docusate, 100 mg b.i.d., Senna, 4 tablets b.i.d., Lactulose or sorbitol, 30 ml q.i.d., and Bisacodyl rectal suppositories, 3-4 after breakfast

For any method or use described above concerning the treatment of platinum-resistant ovarian cancer using EC145 in combination with pegylated liposomal doxorubicin, an additional embodiment is one further comprising administering EC20 to the patient prior to treatment and assessing the patient to have EC20++ status.

In a further embodiment, there is provided a method of selecting a patient for treatment as described in any method or use described above concerning the treatment of platinum-resistant ovarian cancer using EC145 in combination with pegylated liposomal doxorubicin, comprising administering EC20 to the patient prior to treatment and assessing the patient to have EC20++ status.

In a further embodiment, there is provided a pharmaceutical composition comprising EC145 in an aqueous sterile liquid formulation the components of which comprise monobasic sodium phosphate monohydrate, dibasic disodium phosphate dihydrate, sodium chloride, potassium chloride and water for injection.

In a further embodiment, there is provided a dosage unit comprising EC145 drug product for intravenous administration as 2.0 mL of an aqueous sterile liquid formulation, pH 7.4, which dosage unit contains 1.4 mg/mL of EC145. In one embodiment, the above dosage unit is an ampoule, a sealed vial or a prefilled syringe. In another embodiment, the above dosage unit is a sealed vial.

Embodiments of the invention are further described by the following enumerated clauses:

1. A method of determining whether EC145 is indicated for the treatment of a patient with an ovarian tumor or a lung tumor, the method comprising the step of determining whether functionally active folate receptors are present on the tumor of the patient wherein the EC145 is indicated for the treatment of the patient with the tumor if functionally active folate receptors are present on the tumor. 2. The method of clause 1 further comprising the step of administering to the patient EC20 for detection of the functionally active folate receptors. 3. The method of clause 2 further comprising the step of administering to the patient an unlabeled folate prior to administration of the EC20. 4. The method of clause 2 or clause 3 wherein the EC145 is indicated for the treatment of the patient with the tumor if the radioactive signal produced by the EC20 upon binding to the tumor compared to the background radioactive signal produced by the EC20 is indicative of a clinical benefit to the patient. 5. The method of clause 4 wherein the clinical benefit is progression-free survival of the patient. 6. The method of clause 4 wherein the clinical benefit is inhibition of tumor growth. 7. The method of clause 4 wherein the clinical benefit is selected from the group consisting stable disease, a partial response, and a complete response. 8. The method of clause 4 wherein the level of expression of the functionally active folate receptors is quantified based on a tumor to background ratio of the radioactive signal produced by the EC20 to the background radioactive signal. 9. The method of clause 8 wherein the tumor to background ratio is at least about 1.2. 10. The method of clause 8 wherein the tumor to background ratio is at least about 1.3. 11. The method of clause 8 wherein the tumor to background ratio is at least about 1.4. 12. The method of any one of clauses 1 to 11 wherein the tumor is an ovarian tumor. 13. The method of clause 12 wherein the tumor is a platinum-resistant ovarian tumor. 14. The method of any one of clauses 1 to 11 wherein the tumor is a lung tumor. 15. The method of clause 14 wherein the tumor is a non-small cell carcinoma of the lung. 16. The method of any one of clauses 1 to 15 wherein either the EC145, the EC20, or both are in a parenteral dosage form. 17. The method of clause 16 wherein the dosage form is selected from the group consisting of intradermal, subcutaneous, intramuscular, intraperitoneal, intravenous, and intrathecal. 18. The method of any one of clauses 1 to 17 wherein the EC145 is in a composition and wherein the composition further comprises a pharmaceutically acceptable carrier. 19. The method of any one of clauses 2 to 18 wherein the composition comprising the EC20 further comprises a pharmaceutically acceptable carrier. 19a. The method of clause 18 or 19 wherein the pharmaceutically acceptable carrier is a liquid carrier. 19b. The method of clause 19a wherein the liquid carrier is selected from the group consisting of saline, glucose, alcohols, glycols, esters, amides, and a combination thereof. 20. The method of any one of clauses 1 to 19b wherein the EC145 is administered in a therapeutically effective amount. 21. The method of any one of clauses 2 to 20 wherein the EC20 is administered in a therapeutically effective amount. 21a. The method of clause 20 or 21 wherein the effective amount ranges from about 1 ng to about 1 mg per kilogram of body weight. 21b. The method of clause 21a wherein the effective amount ranges from about 100 ng to about 500 μg per kilogram of body weight. 21c. The method of clause 21b wherein the effective amount ranges from about 100 ng to about 50 μg per kilogram of body weight. 21d. The method of any one of clauses 1 to 21c wherein the tumor is a primary tumor. 21e. The method of any one of clauses 1 to 21c wherein the tumor is a metastasized tumor. 21f. The method of any one of clauses 1 to 21e or clauses 24 to 25y wherein EC20 as the folate-radioactive imaging conjugate is replaced by a compound having the formula

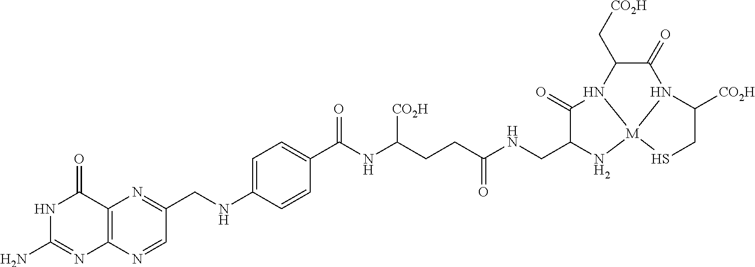

or a pharmaceutically acceptable salt thereof; wherein M is a cation of a radionuclide. 21g. The method of clause 21f wherein the folate-radioactive imaging conjugate is a compound having the formula

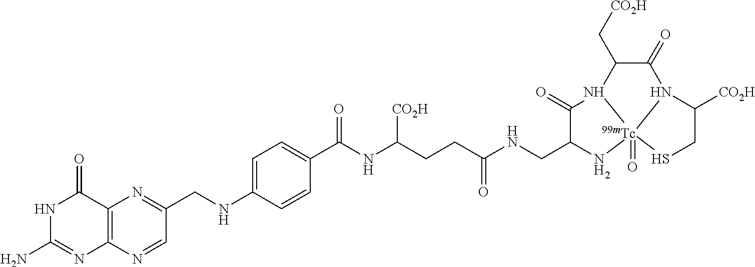

or a pharmaceutically acceptable salt thereof. 21h. The method of clause 21f wherein the folate-radioactive imaging conjugate is a compound having the formula

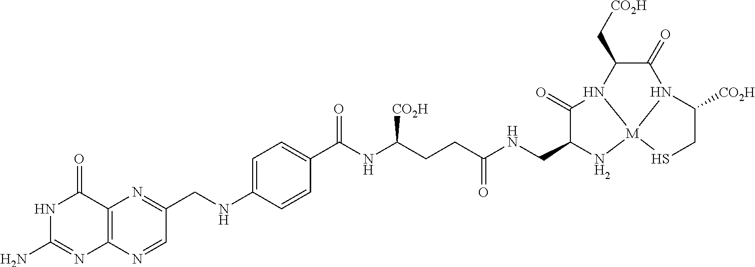

or a pharmaceutically acceptable salt thereof. 21i. The method of clause 21f or 21h wherein M is selected from the group consisting of isotopes of gallium, indium, copper, technetium, and rhenium. 21j. The method of clause 21i wherein M is an isotope of technetium. 21k. The method of clause 21g or 21h wherein the folate-radioactive imaging agent conjugate is radiolabeled using a chelating agent and a reducing agent. 21l. The method of clause 21k wherein the chelating agent is sodium α-_{D}-glucoheptonate. 21m. The method of clause 21k or 21l wherein the reducing agent is tin (II) chloride dihydrate. 21n. The method of any one of clauses 1 to 21m or clauses 24 to 25y further comprising the step of administering to the patient a pegylated liposomal doxorubicin. 22. The method of any one of clauses 1 to 21n further comprising the step of administering to the patient doxorubicin. 23. The method of clause 22 wherein the doxorubicin is in the form of a pegylated liposomal doxorubicin. 24. A method of determining whether EC145 is indicated for the treatment of a patient with an ovarian tumor or a lung tumor, the method comprising the step of administering to the patient a composition comprising EC20, wherein EC145 is indicated for the treatment of the patient with the tumor if the tumor of the patient has functionally active folate receptors wherein the functionally active folate receptors are capable of detection with EC20. 25. The method of clause 24 further comprising the step of administering to the patient an unlabeled folate prior to administration of the EC20. 25a. The method of clause 25 wherein EC145 is indicated for the treatment of the patient with the tumor if the radioactive signal produced by EC20 upon binding to the tumor compared to the background radioactive signal produced by EC20 is indicative of a clinical benefit to the patient. 25b. The method of clause 25a wherein the clinical benefit is progression-free survival of the patient. 25c. The method of clause 25a wherein the clinical benefit is inhibition of tumor growth. 25d. The method of clause 25a wherein the clinical benefit is selected from the group consisting stable disease, a partial response, and a complete response. 25e. The method of clause 25a wherein the level of expression of the functionally active folate receptors is quantified based on a tumor to background ratio of the radioactive signal produced by EC20 to the background radioactive signal. 25f. The method of clause 25e wherein the tumor to background ratio is at least about 1.2. 25g. The method of clause 25e wherein the tumor to background ratio is at least about 1.3. 25h. The method of clause 25e wherein the tumor to background ratio is at least about 1.4. 25i. The method of any one of clauses 24 to 25h wherein the tumor is an ovarian tumor. 25j. The method of clause 25i wherein the tumor is a platinum-resistant ovarian tumor. 25k. The method of any one of clauses 24 to 25h wherein the tumor is a lung tumor. 25l. The method of any one of clauses 24 to 25i wherein the tumor is a non-small cell carcinoma of the lung. 25m. The method of any one of clauses 24 to 25l wherein either EC145, EC20, or both are in a parenteral dosage form. 25n. The method of clause 25m wherein the dosage form is selected from the group consisting of intradermal, subcutaneous, intramuscular, intraperitoneal, intravenous, and intrathecal. 25o. The method of any one of clauses 24 to 25n wherein EC145 is in a composition and wherein the composition further comprises a pharmaceutically acceptable carrier. 25p. The method of any one of clauses 24 to 25o wherein EC20 further comprises a pharmaceutically acceptable carrier. 25q. The method of clause 25o or 25p wherein the pharmaceutically acceptable carrier is a liquid carrier. 25r. The method of clause 25q wherein the liquid carrier is selected from the group consisting of saline, glucose, alcohols, glycols, esters, amides, and a combination thereof. 25s. The method of any one of clauses 24 to 25r wherein EC145 is administered in a therapeutically effective amount. 25t. The method of any one of clauses 24 to 25s wherein EC20 is administered in a therapeutically effective amount. 25u. The method of clause 25s or 25t wherein the effective amount ranges from about 1 ng to about 1 mg per kilogram of body weight. 25v. The method of clause 25u wherein the effective amount ranges from about 100 ng to about 500 μg per kilogram of body weight. 25w. The method of clause 25v wherein the effective amount ranges from about 100 ng to about 50 μg per kilogram of body weight. 25x. The method of any one of clauses 24 to 25w wherein the tumor is a primary tumor. 25y. The method of any one of clauses 24 to 25w wherein the tumor is a metastasized tumor. 26. The method clause 24 or 25 further comprising the step of administering to the patient doxorubicin. 27. The method of clause 26 wherein the doxorubicin is in the form of a pegylated liposomal doxorubicin. 28. A method of predicting a response of an ovarian tumor or a lung tumor of a patient to therapy with EC145, the method comprising the steps of a) administering to the patient EC20 wherein the EC20 produces a radioactive signal; b) quantifying the radioactive signal produced by the EC20 upon binding of the EC20 to the tumor; c) quantifying the background radioactive signal produced by the EC20; d) comparing the radioactive signal produced upon binding of the EC20 to the tumor to the background radioactive signal; and e) predicting the response of the tumor to the therapy based on the comparison. 29. The method of any one of clauses 1 to 28 wherein 15 mg/month of the EC145 is administered. 30. A method of treatment of platinum-resistant ovarian cancer in a patient in need thereof comprising administering a therapeutic amount of EC145 in combination with a therapeutic amount of pegylated liposomal doxorubicin. 31. Use of EC145 in combination with pegylated liposomal doxorubicin for the treatment of platinum-resistant ovarian cancer in a patient. 32. Use of EC145 for the manufacture of a medicament for the treatment in combination with pegylated liposomal doxorubicin of platinum-resistant ovarian cancer in a patient. 33. A method of obtaining a clinical benefit compared to treatment with a therapeutic amount of pegylated liposomal doxorubicin in the treatment of platinum-resistant ovarian cancer in a patient in need thereof comprising administering a a therapeutic amount of EC145 in combination with a therapeutic amount of pegylated liposomal doxorubicin. 34. The method of clause 33 wherein the clinical benefit is progression-free survival. 35. The method of clause 33 wherein the clinical benefit is overall survival. 36. The method or use of any of clauses 30-35 wherein the purity of EC145 is at least 90%. 37. The method or use of any of clauses 30-35 wherein the EC145 is provided in an aqueous sterile liquid formulation the components of which comprise monobasic sodium phosphate monohydrate, dibasic disodium phosphate dihydrate, sodium chloride, potassium chloride and water for injection. 38. The method or use of any of clauses 30-35 wherein the treatment further comprises a bowel regimen. 39. The method or use of any of clauses 30-38 wherein the EC145 is administered as a bolus over about 10 to 20 seconds. 40. The method or use of any of clauses 30-39 further comprising administering EC20 to the patient prior to treatment and assessing the patient to have EC20++ status. 41. A method of selecting a patient for treatment as described in any one of clauses 30-39 comprising administering EC20 to the patient prior to treatment and assessing the patient to have EC20++ status. 42. A pharmaceutical composition comprising EC145 in an aqueous sterile liquid formulation the components of which comprise monobasic sodium phosphate monohydrate, dibasic disodium phosphate dihydrate, sodium chloride, potassium chloride and water for injection. 43. A dosage unit comprising EC145 drug product for intravenous administration as 2.0 mL of an aqueous sterile liquid formulation, pH 7.4, which dosage unit contains 1.4 mg/mL of EC145. 44. The dosage unit of clause 43 which is an ampoule, a sealed vial or a prefilled syringe. 45. The dosage unit of clause 44 which is a sealed vial. 46. A method of determining whether a patient with a tumor has functionally active folate receptors present on the tumor of the patient, the method comprising the step of administering an effective amount of EC20 to the patient for detection of the functionally active folate receptors. 47. The method of clause 46 wherein the tumor is an ovarian tumor or a lung tumor. 48. The method of clauses 46 wherein the tumor is a primary tumor or a metastatic tumor. 49. The method of any one of clauses 1-3, 24-27, or 46-48 wherein the functionally active folate receptors are detected visually. 50. The method of clause 49 wherein the visual detection of functionally active folate receptors is used to determine folate receptor status of the patient. 51. The method of clause 50 wherein the folate receptor status of the patient is selected from the group consisting of EC20++, EC20+, and EC20−. 52. The method of clause 51 wherein the folate receptor status is EC20++. 53. The method of clause 52 wherein treatment with EC145 is indicated. 54. The method of clause 52 wherein EC20++ status correlates with a clinical benefit to the patient. 55. The method of clause 54 wherein the clinical benefit is disease control rate. 56. The method of clause 54 wherein the clinical benefit is overall disease response rate. 57. The method of clause 54 wherein the clinical benefit is overall survival. 58. A method of treatment of a folate receptor expressing epithelial tumor in a patient in need thereof comprising administering a therapeutic amount of EC145 in combination with a therapeutic amount of doxorubicin. 59. The use of EC145 in combination with pegylated liposomal doxorubicin for the treatment of a folate receptor expressing epithelial tumor in a patient. 60. The use of EC145 for the manufacture of a medicament for the treatment in combination with pegylated liposomal doxorubicin of a folate receptor expressing epithelial tumor in a patient. 61. A method of achieving a clinical benefit in the treatment of a folate receptor expressing epithelial tumor in a patient in need thereof comprising administering a therapeutic amount of EC145 in combination with a therapeutic amount of pegylated liposomal doxorubicin. 62. The method of clause 61 in which the clinical benefit is progression-free survival. 63. The method of clause 61 in which the clinical benefit is overall survival. 64. The method or use of any of clauses 58 to 63 wherein the doxorubicin is in the form of a pegylated liposomal doxorubicin. 65. The method or use of any of clauses 58 to 64 wherein the folate receptor expressing epithelial tumor is an ovarian, endometrial or non-small cell lung cancer (NSCLC) tumor. 66. The method or use of clause 65 wherein the folate receptor expressing epithelial tumor is an ovarian tumor. 67. The method or use of clause 64 wherein the folate receptor expressing epithelial tumor is an ovarian, endometrial or non-small cell lung cancer (NSCLC) tumor. 68. The method or use of clause 67 wherein the folate receptor expressing epithelial tumor is an ovarian tumor. 80A. A method of determining whether EC145, or a pharmaceutically acceptable salt thereof, is indicated for the treatment of a patient with an ovarian tumor or a lung tumor, the method comprising the step of administering to the patient a composition comprising a EC20, wherein EC145 is indicated for the treatment of the patient with the tumor if the radioactive signal produced by EC20 upon binding to the tumor compared to the background radioactive signal produced by EC20 is indicative of a clinical benefit to the patient. 80B. A method of selecting a patient with an ovarian tumor or a lung tumor for therapy with EC145, the method comprising the step of determining if functionally active folate receptors are present on the tumor of the patient wherein the patient is selected for therapy with EC145 if functionally active folate receptors are detected on the tumor. 80C. A method of selecting a patient with an ovarian tumor or a lung tumor for therapy with EC145, the method comprising the step of administering to the patient a composition comprising EC20, wherein the patient is selected for the therapy with EC145 if the tumor of the patient has functionally active folate receptors wherein the functionally active folate receptors are capable of detection with EC20. 80D. A method of selecting a patient with an ovarian tumor or a lung tumor for therapy with EC145, the method comprising the step of administering to the patient EC20, wherein the patient is selected for therapy if the radioactive signal produced by EC20 upon binding to the tumor compared to the background radioactive signal produced by EC20 is indicative of a clinical benefit to the patient. 81. The method of clause 80A, 80B, 80C or 80D further comprising the step of administering to the patient an unlabeled folate prior to administration of the folate-radioactive imaging agent conjugate. 82. The method of clause 81 wherein EC145 is indicated for the treatment of the patient with the tumor if the radioactive signal produced by EC20 upon binding to the tumor compared to the background radioactive signal produced by EC20 is indicative of a clinical benefit to the patient. 83. The method of clause 82 wherein the clinical benefit is progression-free survival of the patient. 84. The method of clause 82 wherein the clinical benefit is inhibition of tumor growth. 85. The method of clause 82 wherein the clinical benefit is selected from the group consisting stable disease, a partial response, and a complete response. 86. The method of clause 82 wherein the level of expression of the functionally active folate receptors is quantified based on a tumor to background ratio of the radioactive signal produced by EC20 to the background radioactive signal. 87. The method of clause 86 wherein the tumor to background ratio is at least about 1.2. 88. The method of clause 86 wherein the tumor to background ratio is at least about 1.3. 89. The method of clause 86 wherein the tumor to background ratio is at least about 1.4. 90. The method of any one of clauses 80A, 80B, 80C, 80D to 89 wherein the tumor is an ovarian tumor. 91. The method of clause 90 wherein the tumor is a platinum-resistant ovarian tumor. 92. The method of any one of clauses 80A, 80B, 80C, 80D to 89 wherein the tumor is a lung tumor. 93. The method of any one of the clauses 80A, 80B, 80C, 80D to 89 wherein the tumor is a non-small cell carcinoma of the lung. 94. The method of any one of clauses 80A, 80B, 80C, 80D to 93 wherein either the EC145, EC20, or both are in a parenteral dosage form. 95. The method of clause 94 wherein the dosage form is selected from the group consisting of intradermal, subcutaneous, intramuscular, intraperitoneal, intravenous, and intrathecal. 96. The method of any one of clauses 80A, 80B, 80C, 80D to 95 wherein EC145 is in a composition and wherein the composition further comprises a pharmaceutically acceptable carrier. 97. The method of any one of clauses 80A, 80B, 80C, 80D to 96 wherein the composition comprising EC20 further comprises a pharmaceutically acceptable carrier. 98. The method of clause 96 or 97 wherein the pharmaceutically acceptable carrier is a liquid carrier. 99. The method of clause 98 wherein the liquid carrier is selected from the group consisting of saline, glucose, alcohols, glycols, esters, amides, and a combination thereof.

100. The method of any one of clauses 80A, 80B, 80C, 80D to 99 wherein EC145 is administered in a therapeutically effective amount. 101. The method of any one of clauses 80A, 80B, 80C, 80D to 100 wherein EC20 is administered in a therapeutically effective amount. 102. The method of clause 80A, 80B, 80C, 80D or 101 wherein the effective amount ranges from about 1 ng to about 1 mg per kilogram of body weight. 103. The method of clause 102 wherein the effective amount ranges from about 100 ng to about 500 pg per kilogram of body weight. 104. The method of clause 102 wherein the effective amount ranges from about 100 ng to about 50 μg per kilogram of body weight. 105. The method of any one of clauses 80A, 80B, 80C, 80D to 104 wherein the tumor is a primary tumor. 106. The method of any one of clauses 80A, 80B, 80C, 80D to 104 wherein the tumor is a metastasized tumor. 110. The method of any one of clauses 80A, 80B, 80C, 80D to 109 wherein EC20 as the folate-radioactive imaging conjugate is replaced by a compound having the formula

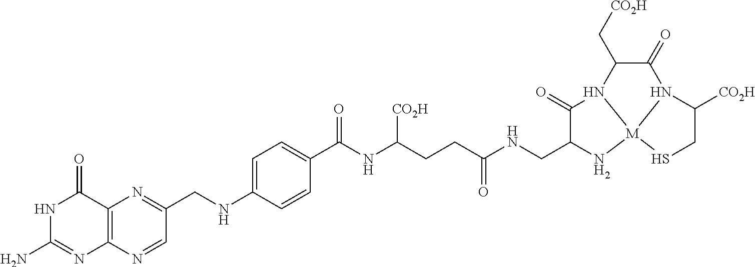

or a pharmaceutically acceptable salt thereof; wherein M is a cation of a radionuclide. 111. The method of clause 110 wherein the folate-radioactive imaging conjugate is a compound having the formula

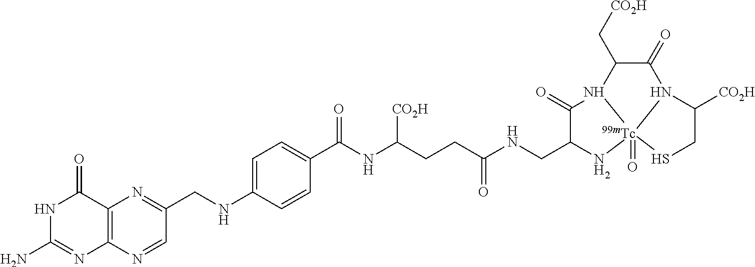

or a pharmaceutically acceptable salt thereof. 112. The method of clause 110 wherein the folate-radioactive imaging conjugate is a compound having the formula

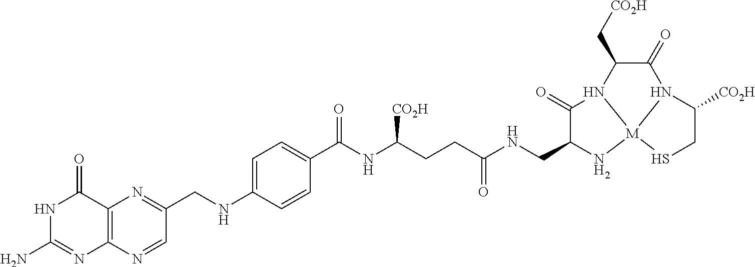

or a pharmaceutically acceptable salt thereof. 113. The method of clause 110 or 112 wherein M is selected from the group consisting of isotopes of gallium, indium, copper, technetium, and rhenium. 114. The method of clause 113 wherein M is an isotope of technetium. 115. The method of clause 111 or 112 wherein the folate-radioactive imaging agent conjugate is radiolabeled using a chelating agent and a reducing agent. 116. The method of clause 115 wherein the chelating agent is sodium α-_{D}-glucoheptonate. 117. The method of clause 115 or 116 wherein the reducing agent is tin (II) chloride dihydrate. 118. The method of any one of clauses 80A, 80B, 80C, 80D to 117 further comprising the step of administering to the patient a pegylated liposomal doxorubicin. 119. The method of any one of clauses 80A, 80B, 80C, 80D to 118 wherein 15 mg/month of the folate-vinca conjugate is administered.

In another embodiment, the methods described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### Example 1

### Materials

N¹⁰-trifluoroacetylpteroic acid was purchased from Eprova AG, Schaffhausen, Switzerland. Peptide synthesis reagents were purchased from NovaBiochem and Bachem. 99mTc Sodium Pertechnetate was supplied by Syncor. [ReO2(en)2]C1 was prepared according to Rouschias (Rouschias, G., Chem. Rev., 74: 531 (1974)). Cellulose plates and DEAE ion exchange plates were purchased from J.T. Baker. DOXIL® was obtained from Ortho Biotech Products, LP, Raritan, N.J.

### Example 2

### Preparation of EC20

EC20 was prepared by a polymer-supported sequential approach using the Fmoc-strategy (Fmoc=9-fluorenylmethyloxycarbonyl; Boc=tert.butyloxycarbonyl; Dap=diaminopropionic acid; DMF=dimethylformamide; DIPEA=diisopropylethylamine). EC20 was synthesized on an acid-sensitive Wang resin loaded with Fmoc-_{L}-Cys(Trt)-OH. Benzotriazole-1-yl-oxy-tris-pyrrolidino-phosphonium hexafluorophosphate (PyBOP) was applied as the activating reagent to ensure efficient coupling using low equivalents of amino acids. Fmoc protecting groups were removed after every coupling step under standard conditions (20% piperidine in DMF). Coupling reactions i.) Fmoc-Asp(OtBu)-OH, PyBop, DIPEA, DMF; ii) Boc-Dap(Fmoc)-OH, PyBop, DIPEA, DMF; iii) Fmoc-D-Glu-OtBu, PyBop, DIPEA, DMF; iv) N¹⁰-TFA-Pte-OH, DIPEA, DMSO. After the last assembly step the peptide was cleaved from the polymeric support by treatment with 92.5% trifluoroacetic acid containing 2.5% ethanedithiol, 2.5% triisopropylsilane and 2.5% deionized water. This reaction also resulted in simultaneous removal of the t-Bu, Boc and trityl protecting groups. Finally, the trifluoroacetyl moiety was removed in aqueous ammonium hydroxide to give EC20.

The EC20 product was purified by HPLC using an Xterra RP18 30×300 mm, 7 μm column (Waters); mobile phase 32 mM HCl (A), MeOH (B); gradient conditions starting with 99% A and 1% B, and reaching 89% A and 11% B in 37 min by a flow rate of 20 mL/min. Under these conditions, EC20 monomer typically eluted at 14.38 min, whereas EC20 disulfide dimer (minor contaminant) eluted at 16.83 min. EC20 analyzed by electrospray-mass spectrometry. Major positive ion peaks (m/z, relative intensity): 746.1, 100; 747.1, 44; 556.8, 32; 570.8, 16.

### Example 3

### Preparation of the Non-Radioactive Reagent Vial and of ^{99m}Tc-EC20.

EC20 kits were used for preparation of the ^{99m}Tc-EC20 radioactive drug substance. Each kit contained a sterile, non-pyrogenic lyophilized mixture of 0.1 mg EC20, 80 mg sodium α-_{D}-glucoheptonate, 80 mg tin (II) chloride dihydrate, and sufficient sodium hydroxide or hydrochloric acid to adjust the pH to 6.8 ±0.2 prior to lyophilization. The lyophilized powder was sealed in a 5 mL vial under an argon atmosphere. The kits were then stored frozen at −20° C. until use or expiration (current shelf life is >2 years). The tin (II) chloride component is present to reduce the added ^{99m}Tc-pertechnetate, while the sodium α-_{D}-glucoheptonate component is present to stabilize the reduced ^{99m}Tc prior to its final chelation to the EC20 compound.

99mTc-EC20 was prepared as follows (i.e., chelation of 99mTc to EC20). First, a boiling water bath containing a partially submerged lead vial shield was prepared. The top of an EC20 vial was swabbed with 70% ethanol to sanitize the surface and the vial was placed in a suitable shielding container. Using a shielded syringe with 27-gauge needle, 1 mL of sterile Sodium Pertechnetate 99mTc Injection (15 to 20 mCi) in 0.9% sodium chloride was injected into the shielded vial. Before removal of the syringe from the vial, a volume of gas from the vial equal to the volume of pertechnetate added was withdrawn in order to normalize the pressure inside the vial. The vial was gently swirled for 30 seconds to ensure complete dissolution of the lyophilized powder. The vial was then placed into the lead shield that was standing in the boiling water bath. The solution was heated for ˜18 minutes and then cooled to room temperature for a minimum of 15 min. This solution can be stored at room temperature (15-25° C.) protected from light, but it should be used within 6 hours of preparation.

### Example 4

### Preparation of EC0119

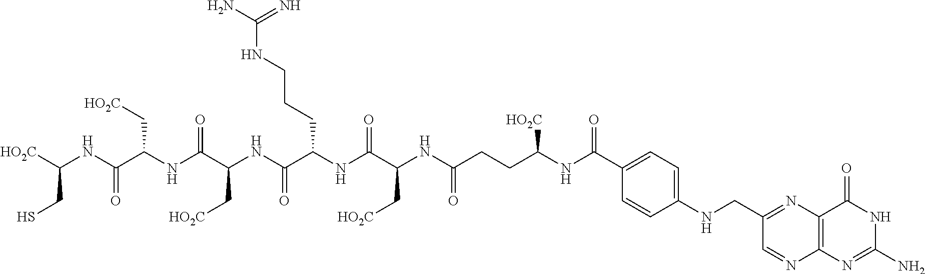

Wang resin bound 4-methoxytrityl (MTT)-protected Cys-NH₂ was reacted according to the following sequence: 1) a. Fmoc-Asp(OtBu)-OH, PyBOP, DIPEA; b. 20% Piperidine/DMF; 2) a. Fmoc-Asp(OtBu)-OH, PyBOP, DIPEA; b. 20% Piperidine/DMF; 3) a. Fmoc-Arg(Pbf)-OH, PyBOP, DIPEA; b. 20% Piperidine/DMF; 4) a. Fmoc-Asp(OtBu)-OH, PyBOP, DIPEA; b. 20% Piperidine/DMF; 5) a. Fmoc-Glu-OtBu, PyBOP, DIPEA; b. 20% Piperidine/DMF; 6) N10-TFA-pteroic acid, PyBOP, DIPEA. The MTT, tBu, and Pbf protecting groups were removed with TFA/H20/TIPS/EDT (92.5:2.5:2.5:2.5), and the TFA protecting group was removed with aqueous NH4OH at pH=9.3. Selected ¹H NMR (D₂O) δ (ppm) 8.68 (s, 1H, FA H-7), 7.57 (d, 2H, J=8.4 Hz, FA H-12 &16), 6.67 (d, 2H, J =9 Hz, FA H-13 &15), 4.40-4.75 (m, 5H), 4.35 (m, 2H), 4.16 (m, 1H), 3.02 (m, 2H), 2.55-2.95 (m, 8H), 2.42 (m, 2H), 2.00-2.30 (m, 2H), 1.55-1.90 (m, 2H), 1.48 (m, 2H); MS (ESI, m+H⁺) 1046.

### Example 5

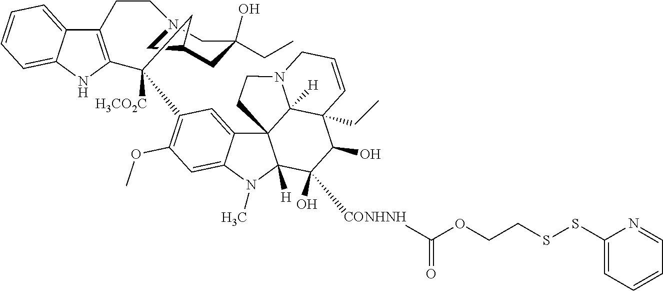

2-[(Benzotriazole-1-yl-(oxycarbonyloxy)-ethyldisulfanyl]-pyridine HCl (601 mg) and 378 μL of DIPEA were sequentially added to a solution of desacetyl vinblastine hydrazide (prepared according to Barnett et al., J. Med. Chem. 21:88-96 (1978), the disclosure of the foregoing is incorporated herein in its entirety by reference. In addition, the entirety of the disclosures of each of the publications cited herein are also incorporated herein by reference) (668 mg) in 5 ml of DCM at 0° C. The reaction was allowed to warm to room temperature and stirred for 3 hours. TLC (15% MeOH in DCM) showed complete conversion. The mixture was purified by silica gel chromatography (1:9 MeOH/DCM). The combined fractions were evaporated, redissolved in DCM and washed with 10% Na2CO3, brine, dried (MgSO4), and evaporated to 550 mg (80%); HPLC-RT 12.651 min., 91% pure, 1H HMR spectrum consistent with the assigned structure, and MS (ESI+): 984.3, 983.3, 982.4, 492.4, 491.9, 141.8.

### Example 6

### Preparation of EC145

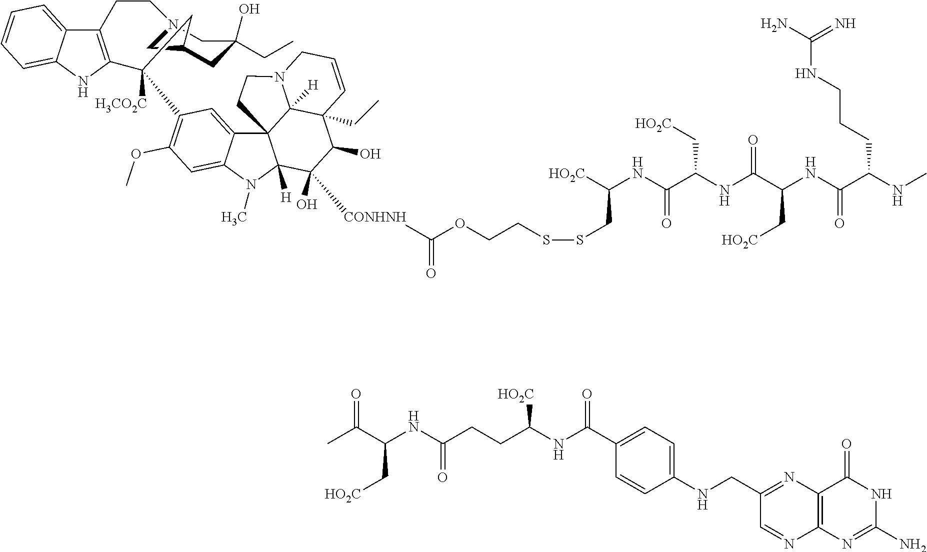

Peptidyl fragment Pte-Glu-Asp-Arg-Asp-Asp-Cys-OH (Example 4) in THF was treated with either the thiosulfonate or pyridyldithio-activated vinblastine (Example 5) as a yellow solution resulting dissolution in 0.1 M NaHCO₃ at pH >6.5 under argon. Lyophilization and HPLC gave a 70% yield; selected ¹H NMR (D₂O) δ 8.67 (s, 1H, FA H-7), 7.50 (br s, 1H, VLB H-11′), 7.30-7.40 (br s, 1H, VLB H-14′), 7.35 (d, 2H, J =7.8 Hz, FA H-12 &16), 7.25 (m, 1H, VLB H-13′), 7.05 (br s, 1H, VLB H-12′), 6.51 (d, 2H, J =8.7 Hz, FA H-13 &15), 6.4 (s, 2H, VLB H-14 & 17), 5.7 (m, 1H, VLB olefin), 5.65 (m, 1H, VLB H-7), 5.5 (d, 1H, VLB olefin), 5.5 (m, 1H, VLB H-6), 4.15(m, 1H, VLB H-8′), 3.82 (s, 3H, VLB C_{18′}—CO₂CH₃), 3.69 (s, 3H, VLB C₁₆—OCH₃), 2.8 (s, 3H, VLB N—CH₃), 1.35 (br s, 1H, VLB H-3′), 1.15 (m, 1H, VLB H-2′), 0.9 (t, 3H, J =7 Hz, VLB H-21′), 0.55 (t, 3H, J =6.9 Hz, VLB H-21); LCMS (ESI, m+H⁺) 1918.

### Example 7

### In Vitro Drug-Drug Combination Assay with EC145 and Doxorubicin

On day one, KB tumor cells were trypsinized, suspended in folate deficient-RPMI (FDRPMI)+5% fetal bovine serum, and counted using a hemacytometer. The cell suspension was diluted to a final concentration of 0.5×10⁵ cells/mL and the diluted suspension used to load six 24-well plates with 1 mL of cell suspension per well. The wells were then divided into test groups with four replicates per sample and allowed to attach to the plate overnight at 37° C., 5% CO₂.

On day two, EC145 and doxorubicin concentrations were prepared from 0.731 mM and 2.9 mM sterile stock solutions, respectively, at 2× the final concentrations and then combined in their corresponding wells with either FDRPMI or the alternate drug in a final volume of 500 μL. The final concentration of EC145 in each individual well was 0 nM, 2 nM, 4 nM, 8 nM, 16 nM, or 32 nM. The final concentration of doxorubicin in each individual well was 0 nM, 12.5 nM, 25 nM, 50 nM, 100 nM, or 200 nM. Four replicates of each of the 36 combinations of EC145 concentration and doxorubicin concentration were tested. Samples containing EC145 were incubated for two hours, replaced with either FDRPMI or the appropriate concentrations of doxorubicin and then incubated a total of 72 hours. Samples of doxorubicin only were incubated for 72 hours uninterrupted. Afterwards, spent incubation medium in each well was replaced with 500 μL of 1 μCi/mL ³H-thymidine in FDRPMI; cells were incubated for four additional hours. After the incubation, the labeling solution was aspirated and the cells were washed twice with PBS. 500 μL of 10% trichloroacetic acid (TCA) was then added to each well, and the plates were stored at 4° C. until they were processed.

Cells were processed by aspirating TCA and adding 500 μL of 0.25 M NaOH. 450 μL of sample from each well was then transferred to individually-labeled liquid scintillation vials, vortexed with 3 mL of Ecolite cocktail, and then counted in a liquid scintillation counter. CPM results were then tabulated and percent control values calculated.

### Isobologram-Drug Synergy Method

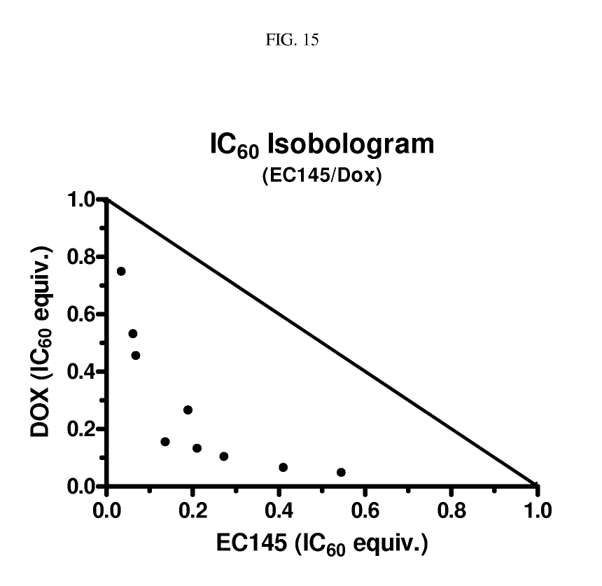

Drug synergy was determined by the isobologram method. In this method IC₆₀ values are forecast from the percent of control values. These data can be graphed as nM values or as equivalents by setting the IC₆₀ of each single agent equal to 1 and all combinational IC₆₀s as a fraction thereof. Combination data points that fall on the line represent an additive drug-drug interaction, whereas data points that fall below or above the line represent synergism or antagonism, respectively. As show in graph in FIG. 15, IC₆₀ values for EC145/doxorubicin combinations fell well below the line, suggesting that EC145 and doxorubicin have a strong synergistic relationship in KB cells.

### Example 8

### Study of EC145 and DOXIL® (PDL) Alone or in Combination in Balb/c− Mice bearing Subcutaneous M109 Tumors Maintained on Folate Deficient Diet

Balb/c-female mice purchased from Harlan (Indianapolis, Ind.) were housed (5 animals/cage) in standard polycarbonate shoebox cages with sani-chips bedding and wire top. The cages were replaced with clean cages every two weeks. The animals were housed throughout the study period in an environmentally controlled room. The room temperature settings ranged from 70° F. to 74° F. The relative humidity of the room ranged from 30% to 70%. Light timers were set to provide a 12-hour light/12-hour dark photoperiod. The animals were observed daily for health.

The animals were initially fed Test Diet #00434 produced by Harlan Teklad (Madison, Wis.). Beginning one week after dosing, the animals were switched to Standard Rodent Diet PMI 5000 manufactured by PMI Labdiet (Richmond, Ind.). The animal feed and drinking water were provided ad libitum throughout the study period.

Tumor implantation

M109 (Madison-109 lung carcinoma cells) tumor cells were grown in folate-deficient RPMI 1640 with 5% FBS at 37° C. in a 5% CO₂ humidified atmosphere. M109 tumor cells (1×10⁶ cells per animal) were inoculated subcutaneously 9 days post start of the folate deficient diet. Mice were dosed after the tumors reached between 70-100 mm³.

### Preparation of Dosing Drug Solutions and Dosing

Dosing solutions were prepared by weighing appropriate amounts of each compound, reconstituting/dissolving in PBS (pH 7.4), sterile filtering the drug solution through a 0.22 μm PVDF syringe filter, and freezing aliquots for each day of dosing at −20° C. Doses were administered i.v. in a volume of 200 μL.

### Evaluation

Tumor size was monitored and body weight measured 3 times/week. Attention was given to gross animal morphology and behavior. Euthanasia was performed if the mice lost >20% of weight or when the tumors reached a size of 1500 mm³. Euthanasia was also performed at the researcher's discretion if mice lost a lot of weight in a short duration or when mice were approaching moribund conditions.

### Results and Conclusions

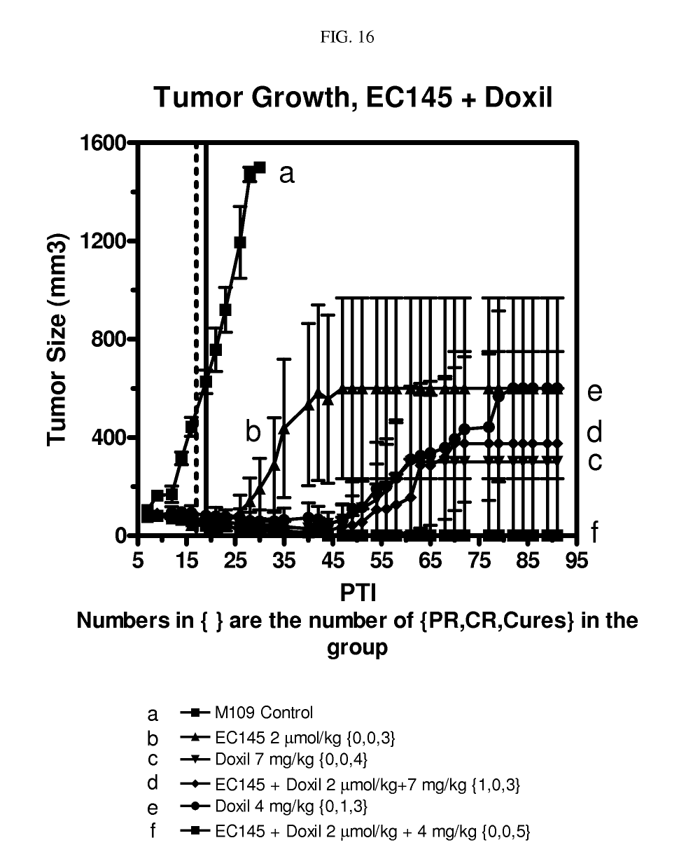

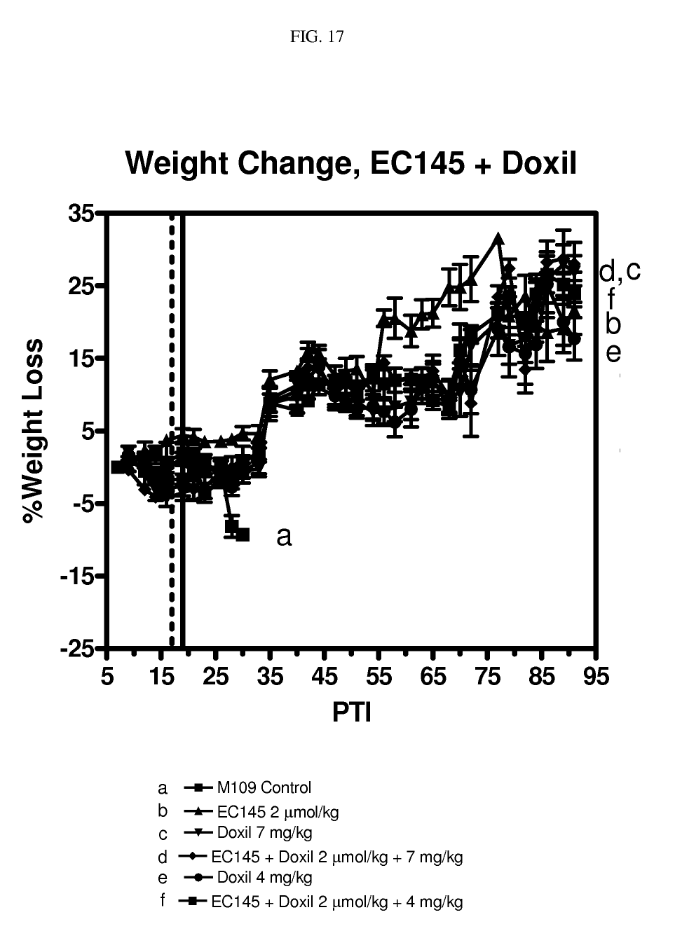

The effects on tumor growth and responses (PR=partial response, CR=complete response, Cures) are illustrated in FIG. 16, and the effects on weight change are illustrated in FIG. 17, respectively, for the following groups: (a) M109 control; (b) EC145, 2 μmol/kg; (c) DOXIL, 7 mg/kg; (d) EC145, 2 μmol/kg+DOXIL, 7 mg/kg; (e) DOXIL, 4 mg/kg; and (f) EC145, 2 μmol/kg+DOXIL, 4 mg/kg. The results for each group are further described below:

(b) EC0145 at 2 μmol/kg, TIW×2 doses displayed good anti-tumor effect with 3 of the 5 mice cured of visible tumor. Mice in this group had no weight loss during the dosing period.

(c) Doxil at 7 mg/kg, q7d×2 doses displayed marked anti-tumor effect with 4 of 5 cures. Mice in this group did experience slight weight loss (2-8%) during the dosing period.

(d) EC0145 at 2 μmol/kg, TIW×2 combined with Doxil at 7 mg/kg, q7d×2 doses also displayed good anti-tumor effect with 3 of 5 cures. Mice in this group had slight weight loss (1-6%) during the dosing period. One animal died during dose 5 due to unknown causes. This animal had a partial response at this time.

(e) Doxil at 4 mg/kg, q7d×3 doses displayed marked anti-tumor effect with ⅕ complete response and ⅗ cures. Three mice in this group also experienced prolonged weight loss (2-10%) upon completion of dosing, but eventually regained their weights.

(f) EC0145 at 2 μmol/kg, TIW×2, combined with Doxil at 4 mg/kg, q7d×3 doses displayed excellent anti-tumor effect with 5 of 5 cures. Mice in this group had a mild weight loss of 0 to 5% during and after the dosing period.

### Example 14

### Study of EC145 in Patients with Advanced Ovarian and Endometrial Cancers

The protocol for this study (EC-FV-02) is summarized at http://www.clinicaltrials.gov/ct2/show/NCT00507741?term=Endocyte&rank=3 which is incorporated by reference herein.

### Example 15

### Study of EC145 in Patients with Progressive Adenocarcinoma of the Lung

The protocol for this study (EC-FV-03) is summarized at http://www.clinicaltrials.gov/ct2/show/NCT00511485?term=Endocyte&rank=7 which is incorporated by reference herein.

### Example 16

### Tumor Imaging Protocol Using ^{99m}Tc-EC20

^{99m}Tc-EC20 dosing and imaging was performed within 21 days but not less than 7 days prior to the initiation of EC145 treatment.

Administration of ^{99m}Tc-EC20.

Prior to the ^{99m}Tc-EC20 imaging procedure, patients received one IV injection of 0.5 mg of folic acid, followed, within 1 to 3 minutes, by a 1 to 2 mL injection of 0.1 mg of EC20 labeled with 20 to 25 mCi of technetium-99m. If possible, folic acid supplements were discontinued within a week of administration of ^{99m}Tc-EC20.

Folic acid was administered by IV injection approximately 1 to 3 minutes prior to administration of ^{99m}Tc-EC20. Folic acid was injected as a slow IV push followed by 5 to 10 mL of normal saline via a free-flowing indwelling IV catheter in an upper extremity vein (e.g., antecubital fossa) or appropriate indwelling IV access.

^{99m}Tc-EC20 was administered in a volume of approximately 1 to 2 mL via a free flowing indwelling IV catheter. ^{99m}Tc-EC20 may be administered in the same line as the folic acid. ^{99m}Tc-EC20 was administered over a period of approximately 30 seconds, followed by 5 to 10 mL injections of normal saline. The injected radioactive dose was between 20 and 25 mCi.

### Image Acquisition

Approximately 1 to 2 hours post-injection of ^{99m}Tc-EC20, mid-thigh to head, anterior and posterior planar images were acquired. Immediately after planar images were acquired, SPECT (or SPECT/CT) images of the anatomic region known to contain the tumor as identified by the patient's conventional image were acquired. If the anatomic region containing the tumor was not previously identified, SPECT (or SPECT/CT) images of the chest/abdomen and abdomen/pelvis were acquired.

### Planar Imaging

A mid-thigh to head, anterior and posterior planar images were acquired according to the following required parameters: 1.) Imaging Area: Mid-thigh to head 2.) Camera: Dual or triple-headed detector large field of view (FOV) LEHR parallel-hole collimators, 3.) Matrix: 256×1024 minimum, 4.) Energy Window: 15%-20%, 5.) Energy keV: 140, and 6.) Scan Speed: 8-10 cm/minute.

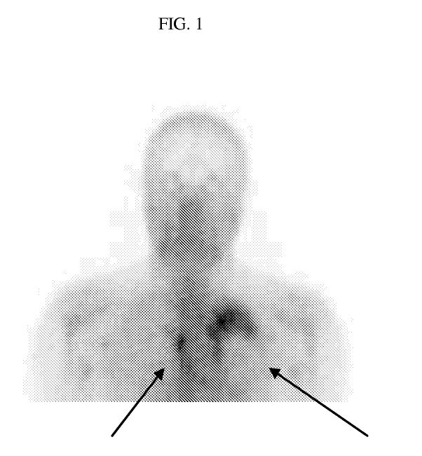

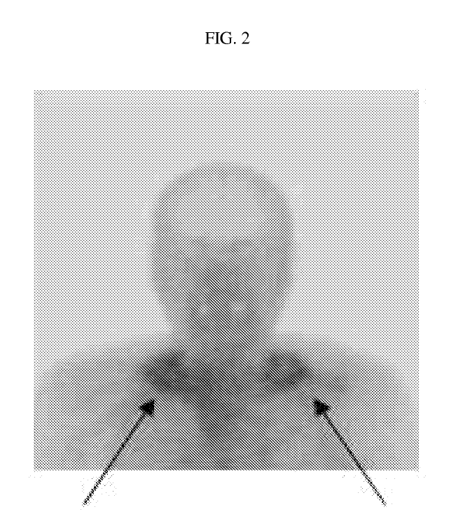

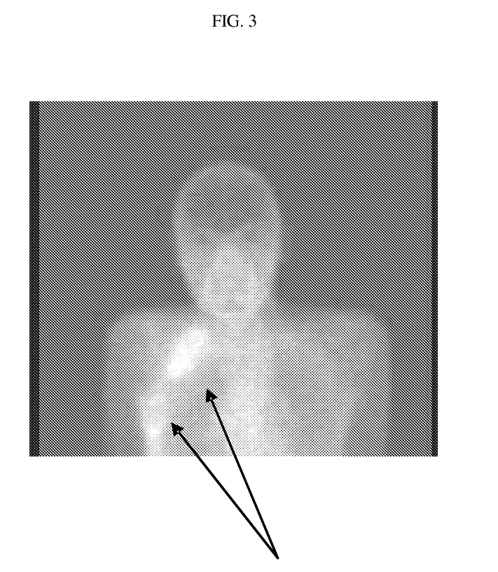

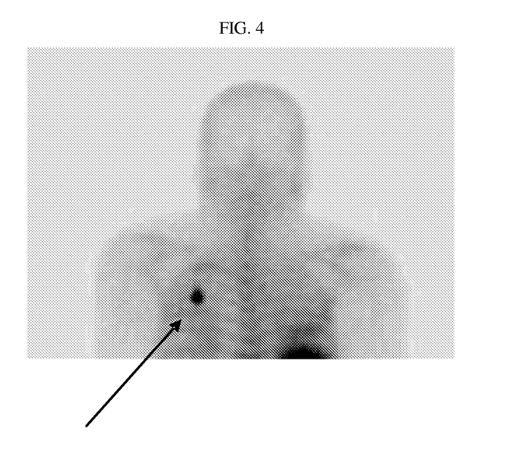

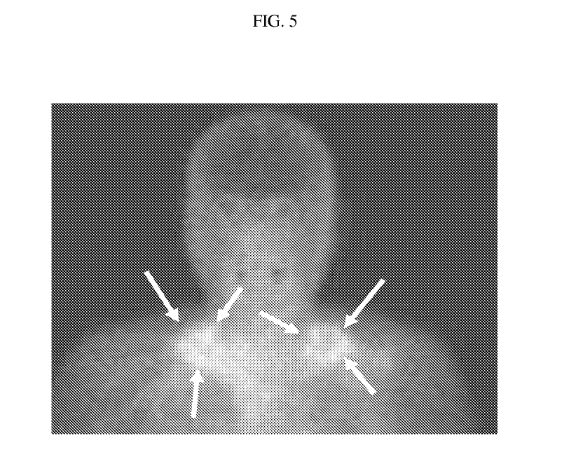

Representative planar images are shown in FIGS. 1, 2, 3, 4, and 5. Tumor locations are indicated by the arrows added to the images.

### SPECT Imaging

For optimal imaging of the body, the arms were elevated over the head if tolerated by the patient. For optimal imaging of the head and neck, the arms were positioned along the sides. Images of the region known to contain the target lesion(s) as identified by the patient's conventional image were obtained, immediately after the planar images were acquired.

If all target lesions are not in the FOV for the first image acquisition, additional imaging was performed to obtain an image of all target lesions. SPECT/CT may be used in place of plain SPECT using the attenuation correction parameters listed below. Data was reconstructed at the highest pixel resolution using iterative reconstruction (a minimum of 6 iterations is recommended). SPECT is reconstructed into 3 orthogonal planes: transverse, sagittal, and coronal.

Images of the region known to contain the target lesion(s) were acquired according to the following required parameters: 1.) Camera: Dual or triple-headed detector large FOV LEHR parallel-hole collimators 2.) Total Projections: 120-128 3.) Matrix: 128×128 4.) Orbit Type: Circular or Elliptical 5.) Orbit: 180 degrees per head with a dual detector camera OR 120 degrees per head with a triple detector camera 6.) Time per Stop: 40 seconds/stop 7.) Total Number of Stops: 60 to 64 projections per head for a dual-head camera or 40 to 43 projections per head for a triple-head camera 8.) Energy Window: 15% -20% 9.) Energy keV: 140

### SPECT/CT Imaging

Acquisition of CT images using SPECT/CT equipment followed the Society of Nuclear Medicine Procedure Guideline for SPECT/CT Imaging.

CT images were acquired only for the purposes of attenuation correction/anatomic localization (AC/AL) unless the CT component of the combined SPECT/CT system was capable of providing diagnostic images with image quality and resolution that met or exceeded that of available dedicated diagnostic CT equipment.

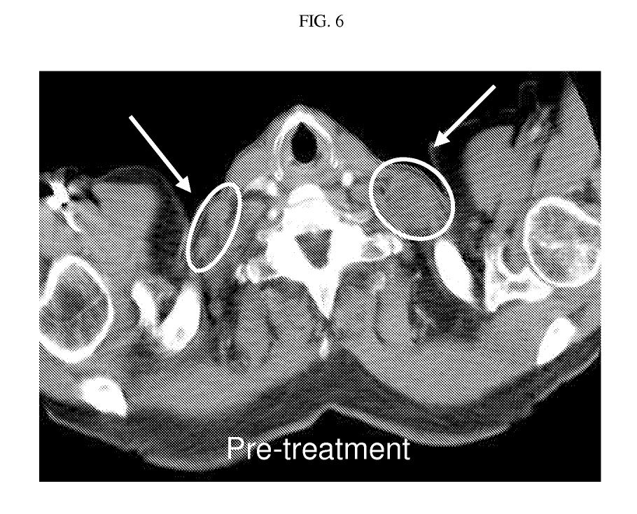

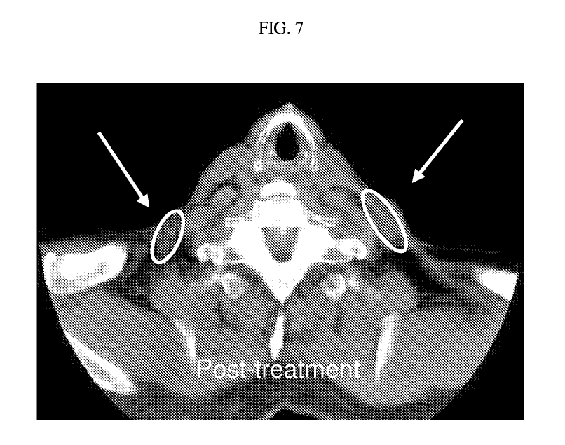

CT images were acquired using a 256×256 minimum matrix, a maximum 7.5-mm slice thickness, spiral acquisition, at 140 kVp and 80 mA during normal (tidal) respiration. AC/AL CT sinograms were reconstructed with filtered backprojection at the full FOV. The filtered back projection was either 2-dimensional after appropriate portions of the spiral CT data were collected into axial or tilted planes or fully dimensional. Standard kernels were used for attenuation correction. CT may be reformatted into three orthogonal planes: transverse, sagittal, and coronal. See FIGS. 6 and 7.

### Example 17

### Tumor-to-Background Measurement

### EC20 3-Coded Scale

For both the planar images and the SPECT/CT or SPECT images, the nuclear medicine physician coded the intensity of uptake for each target lesion (e.g., T1, T2, T3). If a lesion was not in the SPECT region, it was coded as not imaged.
1. No uptake: No uptake as compared to background.
2. Mild Uptake: Uptake increased slightly as compared to background.
3. Marked Uptake: Uptake significantly increased as compared to background.

For any area showing uptake that does not correspond to a radiographic abnormality, including organs, the nuclear medicine physician documented the location and coded the intensity of uptake using the same 3-coded scale.

### Tumor-to-Background Ratio

SPECT images were analyzed semi-quantitatively using a tumor-to-background (T/B) ratio. For each target lesion (e.g., T1, T2, T3), a region of interest (ROI) was drawn over the areas of maximum activity within the lesion that corresponds to the radiographic abnormality. The region was used to provide the tumor measurement. For each target lesion, an ROI of the corresponding minor image location available in the normal appearing contralateral area was drawn. If the region was an area showing uptake, an ROI of normal tissue adjacent to the lesion was drawn. This region was used to provide the background measurement.

For any area showing uptake that did not correspond to a radiographic abnormality, including organs, the location was documented and an ROI was drawn over the area of maximum activity within the area of uptake. An ROI of the corresponding mirror image location available in the normal-appearing contralateral anatomy was drawn. If the contralateral site was an area showing uptake, an ROI of normal tissue adjacent to the lesion was drawn.

The tumor-to-normal tissue background (T/B) ratio for each lesion is calculated from the measurement derived from each ROI pair.

### Example 18

### Patient Selection and Treatment Regimen with EC145, Lung Tumor

### Patient Selection Criteria

Patients had advanced, progressive, adenocarcinoma of the lung, had previously received two or more cytotoxic agent containing chemotherapeutic regimens, had a performance status of 0 to 2 on the Eastern Cooperative Oncology Group (ECOG) scale, were at least 4 weeks from prior therapy and recovered (to baseline) from associated acute toxicities. Patients also had radiographic evidence of measurable disease and more than one area of tumor that was also identified as “EC20 positive” (i.e., a tumor-to-background ratio≧1.2).

### Treatment Regimen

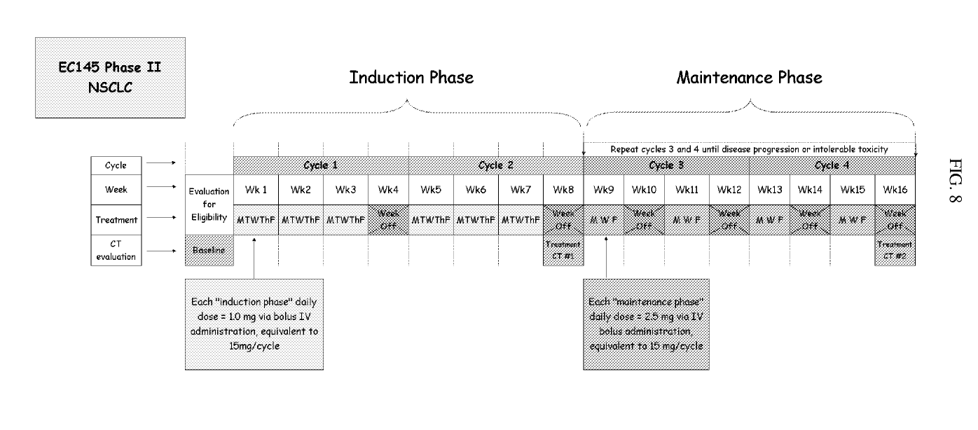

EC145 (1 mg/injection) was administered intravenously as a bolus injection on M, T, W, Th, and F during weeks 1 to 3 in each 4-week cycle. No treatment was administered in week 4 (total dose administered to the patient was 15 mg/month). This cycle was repeated twice in the induction phase. This phase was followed by the maintenance phase which consisted of injections of 2.5 mg/injection, administered intravenously as a bolus injection, on M, W, F of weeks 1 and 3 of the 4-week cycle. No treatment was administered in weeks 2 and 4 (total dose administered to the patient was 15 mg/month). See FIG. 8 for a graphical description of the dosing schedule.

### Example 19

### Treatment of Patients with EC145 Combined with ^{99m}Tc-EC20 Monitoring

Patients were screened prior to the beginning of administration of EC145 using the methods of Example 16. EC145 was administered to the patients following the regimen described in Example 18.

TABLE 1

| | Population | |
|---|---|---|
| | Fully eligible patients (all | All treated patients (includes |
| | patients with screening CT | 11 pts with screening CT > 28 |
| | within 28 days of onset of | days (range 29-39 d) before |
| | EC145 therapy) | receiving EC145) |
| Endpoint | n = 29 | n = 42 |
| Clinical Benefit | 31% (9) | 25% (11) |
| Disease Control | 41% (12) | 35.7% (15) |
| Rate at 8 weeks | | |
| RECIST | 1 PR | 1 PR |
| response | | |

Table 1 shows that patients treated with EC145 derived clinical benefit (defined as the ability to receive 4 or more cycles of therapy) at rates greater than 20%, thus the primary endpoint for the study was achieved.
RECIST, Complete Response (CR): Disappearance of all target lesions
RECIST, Partial Response (PR): At least a 30% decrease in the sum of the longest dimension (LD) of target lesions, taking as reference the baseline sum LD
RECIST, Stable Disease (SD): Neither sufficient shrinkage to qualify for PR nor sufficient increase to qualify for PD, taking as reference the smallest sum LD since the treatment started
RECIST, Progressive Disease (PD): At least a 20% increase in the sum of the LD of target lesions, taking as reference the smallest sum LD recorded since the treatment started or the appearance of one or more new lesions

TABLE 2

| | Population | |
|---|---|---|
| | All patients receiving | 3^{rd}/4^{th} line patients with |
| | EC145 as 3^{rd} or 4^{th} line | 100% of tumors showing |
| | IV therapy | EC20 uptake |
| Endpoint | n = 20 | n = 11 |
| Clinical Benefit | 40% (8) | 45% (5) |
| Disease Control | 50% (10) | 63.6% (7) |
| Rate at 8 weeks | | |
| RECIST response | 1 PR | 1 PR |

The primary endpoint criterion requires response rate of ≧20%

Subset analysis of patients receiving EC145 as 3^{rd} or 4^{th} line therapy indicates a clinical benefit rate of 40%.

In patients with EC20 uptake (indicating FR expression) in all tumors, the clinical benefit rate increases to 45%.

TABLE 3

| | Population | |
|---|---|---|
| | | 3^{rd}/4^{th} line patients with |
| | 3^{rd}/4^{th} line patients with | heterogenous EC20 uptake |
| | 100% of tumors | (i.e., at least one EC20- |
| | showing EC20 uptake | negative tumor mass) |
| Endpoint | n = 11 | n = 6 |
| Clinical Benefit | 45% (5) | 33% (2) |
| Disease Control | 63.6% (7) | 33% (2) |
| Rate at 8 weeks | | |
| RECIST response | 1 PR | |

The primary endpoint criterion requires response rate of≧20%

### Example 20

### Treatment Regimen with EC145 and DOXIL® (PDL), Ovarian Cancer

### Treatment Regimen (EC145 and PLD)

On the days on which subjects receive EC145 and pegylated liposomal doxorubicin (PLD), EC145 was administered at least 45 minutes prior to the administration of PLD. After the EC145 is administered, the IV hub was flushed, and when at least 45 minutes had elapsed, the PLD was administered via the same IV hub used for administering EC145.

### EC145

EC145 was administered through an IV line (peripheral or indwelling catheters are acceptable) as a bolus injection over approximately 10 to 20 seconds. EC145 was not mixed with any other drug solution during administration and the IV hub was flushed with approximately 10 cc of sterile normal saline solution (or a flush amount per institutional standard of care) both before and immediately after administration of EC145. EC145 (2.5 mg) was administered on Monday, Wednesday, and Friday of weeks 1 and 3 of each 4-week cycle. No therapy was administered during weeks 2 and 4. The schedule for each subsequent cycle after cycle 1 was identical to that of the first cycle.

### Calculation and Delivery of PLD Dose

PLD was administered IV at a dose of 50 mg/m². For subjects whose measured body weight was greater than their ideal body weight, the dose of PLD was calculated on the basis of ideal body weight (IBW). After the subject's height in centimeters was determined, IBW was calculated as follows:

IBW=45.5 kg+0.9 kg for each centimeter over 152 cm

Body surface area (BSA) in square meters is then calculated as follows:

BSA (m²)=([height (cm)×IBW (kg)]/3600)^{1/2}, alternatively,

BSA (m²)=the square root of ([height (cm)×IBW (kg)]/3600)

PLD was administered at a rate of 1 mg/min to minimize the risk of infusion reactions. If no infusion-related adverse reactions were observed, the rate of the infusion was increased to complete administration of the drug over 1 hour. The risk of cardiotoxicity increased with the cumulative dose of doxorubicin. The recommended lifetime maximum dosage of conventional doxorubicin was as follows:

Adults<550 mg/m²

Adults>70 years of age Consider cumulative dose of<300 mg/m² (Cancer Chemotherapy Manual, published by Walters Kluwer Health© University of Utah, August 2006)

The subject received a dose of PLD once every 28 days on day 1 (for a recommended minimum of 4 courses) until the maximum allowable cumulative dose of 550 mg/m2 was attained as long as the subject did not exhibit disease progression, did not show evidence of cardiotoxicity, and continued to tolerate treatment.

The pegylated liposomal doxorubicin used in this study is a mixture comprising liposomes containing doxorubicin or a salt thereof where the liposomes comprise a polyethylene glycol modified surface. In illustrative examples, the pegylated liposomal doxorubicin was DOXIL®. DOXIL® is doxorubicin HCl encapsulated in STEALTH® liposome carriers. STEALTH® liposome carriers were composed of N-(carbonyl-methoxypolyethylene glycol 2000)-1,2-distearoyl-sn-glycero-3-phosphoethanolamine sodium salt (MPEG-DSPE), 3.19 mg/mL; fully hydrogenated soy phosphatidylcholine (HSPC), 9.58 mg/mL; and cholesterol, 3.19 mg/mL. Each mL also contained ammonium sulfate, approximately 2 mg; histidine as a buffer; hydrochloric acid and/or sodium hydroxide for pH control; and sucrose to maintain isotonicity. Greater than 90% of the drug was encapsulated in the STEALTH® liposomes.

### Example 21

### Tumor response in NSCLC and Ovarian Cancer evaluable lesions

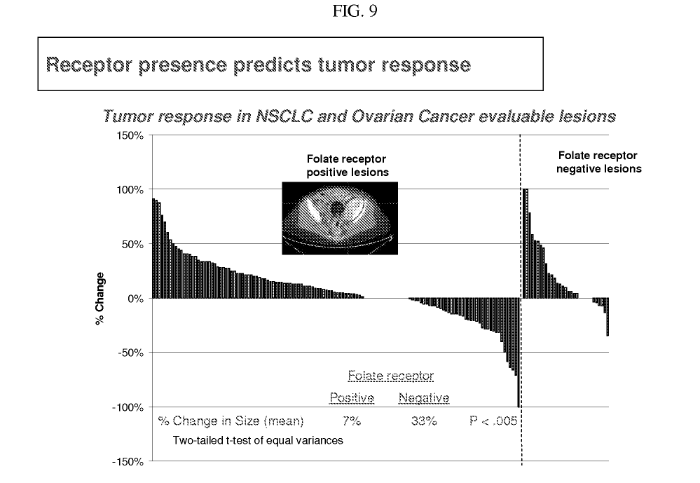

Using the imaging methods described in Example 16, tumors were imaged during the treatment described in Example 18 or Example 19. The percentage size change for each imaged tumor is shown in FIG. 9. The data show that folate-receptor positive tumors (selected based on use of the ^{99m}Tc-E20 imaging method described herein) have a mean increase in size of only 7% compared to the mean increase in size of 33% shown for the folate-receptor negative tumors (lesions).

While certain embodiments of the present invention have been described and/or exemplified above, it is contemplated that considerable variation and modification thereof are possible. Accordingly, the present invention is not limited to the particular embodiments described and/or exemplified herein.

### Example 22

EC145 for Injection (EC145 Drug Product) Specifications and
Representative Results.
Storage/Handling Instructions: Store at −20° C. ± 5° C., protect from light

| Test | Specifications | Results |
|---|---|---|
| Appearance | Yellow solution | Yellow Solution |
| Identity | M + 2H⁺ at 959.4 ± 1.0 | 959.5 |
| Purity | ≧90.0% | 95.6% |
| Largest Individual | <7% | 0.4% |
| Oxidation Product | | |
| Largest Individual | <4% | 1.0% |
| Related Substance | | |
| EC145 Content | 90-110% label content | 105% |
| Total Related Substances | ≦10% | 4.4% |
| pH | 6.7-7.8 | 7.6 |
| Osmolality | TBM¹ | 282 mOsmol |
| Sterility | pass | Pass |
| Endotoxin | NMT 119 EU/vial | <11 EU/vial |
| Particulate Matter | ≧10 micron NMT 6000/vial | 108/vial |
| | ≧25 micron NMT 600/vial | 5/vial |
| Residual Solvents | ≦1% | <1% |
| Volume in Container | ≧1.8mL in Vial | >2.0 mL/vial |

¹TBM is to be monitored

### Example 23

EC145 drug product (DP) for intravenous (IV) administration is provided as 2.0 mL of an aqueous sterile liquid formulation, pH 7.4, in single-use clear glass vials with Flurotech™-coated rubber stoppers and is stored frozen under argon. Each vial contains 1.4 mg/mL of EC145. The quantitative composition of the drug product is shown in the table below. Single vials are used to provide a 2.5 mg bolus dose of EC145.

EC145 Drug Product Components

| | | | Amount per vial |
|---|---|---|---|
| | Function | Grade | (mg) |
| EC145 | Active | In-house | 2.8 |
| Sodium phosphate, | pH control | USP | 1.1 |
| monobasic monohydrate | tonicity | | |
| Disodium phosphate, | pH control | USP | 2.14 |
| dibasic dihydrate | Tonicity | | |
| Sodium chloride | Tonicity | USP | 16.12 |
| Potassium chloride | Tonicity | USP | 0.4 |
| Water for Injection | Solvent | WFI | QS to 2.0 mL |

### Example 24

### Representative Bowel Regimen for Treatment/Prevention of Constipation

Constipation/ileus was noted as a potentially serious adverse event in the Phase I trial of EC145, especially in those subjects who received concomitant opioid analgesia.

A suggested progressive bowel regimen (Modified from Carney Mont., Meier Del. Palliative care and end-of-life issues. Anaesthesiol Clin North America 2000;18:183.) for subjects who receive therapy with EC145 should parallel that used for subjects who receive opioid therapy in which clinicians can progress through higher steps until an effective regimen is found:

Step 1: Docusate, 100 mg twice daily (b.i.d.) and Senna, 1 tablet once daily (q.d.) or b.i.d.

Step 2: Docusate, 100 mg b.i.d., Senna, 2 tablets b.i.d., and Bisacodyl rectal suppositories, 1-2 after breakfast.

Step 3: Docusate, 100 mg b.i.d., Senna, 3 tablets b.i.d., and Bisacodyl rectal suppositories, 3-4 after breakfast.

Step 4: Docusate, 100 mg b.i.d., Senna, 4 tablets b.i.d., Lactulose or sorbitol, 15 mL b.i.d., and Bisacodyl rectal suppositories, 3-4 after breakfast.

Step 5: Docusate, 100 mg b.i.d., Senna, 4 tablets b.i.d., Lactulose or sorbitol, 30 ml b.i.d., and Bisacodyl rectal suppositories, 3-4 after breakfast.

Step 6: Docusate, 100 mg b.i.d., Senna, 4 tablets b.i.d., Lactulose or sorbitol, 30 ml q.i.d., and Bisacodyl rectal suppositories, 3-4 after breakfast.

### Example 25

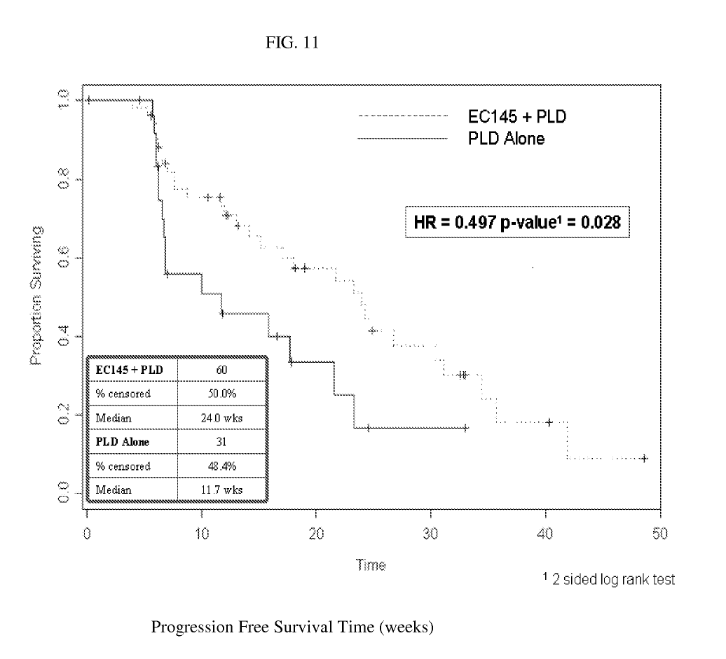

PRECEDENT: A randomized phase II trial comparing EC145 and pegylated liposomal doxorubicin (PLD) in combination, versus PLD alone, in subjects with platinum-resistant ovarian cancer (EC-FV-04).
Background: EC145, a conjugate of folic acid and desacetylvinblastine hydrazide binds with high affinity to the folate receptor (FR), found on >90% of epithelial ovarian cancers. This example reports interim data on an international randomized, phase 2 study of EC145+PLD compared with PLD alone, in women with platinum-resistant ovarian cancer. An independent Data Safety Monitoring Board (DSMB) has conducted a pre-specified interim analysis on PFS and safety with results reported herein.
Methods: Women≧18 with ECOG PS of 0-2 and<2 prior systemic cytotoxic regimens were randomized to receive EC145 (2.5 mg IV weeks 1 and 3)+PLD (50 mg/m² IBW IV q 28 days) or PLD (50 mg/m² IBW IV q 28 days) alone until progression or death.
Results: The interim analysis occurred after the 46th event out of a planned study total of 95 progressions or deaths. Demographic characteristics at screening such as age, cancer type, residual tumor after debulking, prior therapy, CA-125 and time from diagnosis were balanced between arms. RECIST mean sum tumor length was longer for the combination arm (122.7 mm vs. 81.3 mm). There was no statistical difference between study arms with regard to total adverse events, serious adverse events, or the number of subjects reporting at least one treatment-emergent drug-related serious adverse event resulting in discontinuation. The table below displays the results of the interim analysis of PFS and the Kaplan Meier curve can be found in FIG. 11.

| Patient | EC145 + PLD | PLD | | |
|---|---|---|---|---|
| Population | PFS (weeks) | PFS (weeks) | Hazard Ratio | P-value |
| EC-FV-04 Study | 24.0 | 11.7 | 0.497 | 0.014 |

At the interim, there is also a trend toward benefit in overall survival for the combination arm with HR=0.425 (P-value of 0.064).
Conclusions: Results indicate a greater than doubling in median PFS for women with platinum-resistant ovarian cancer receiving EC145 and PLD over those receiving PLD alone. These interim data suggest that EC145 and PLD is the first combination to show a statistically significant increase in progression free survival over standard therapy in women with platinum-resistant ovarian cancer.

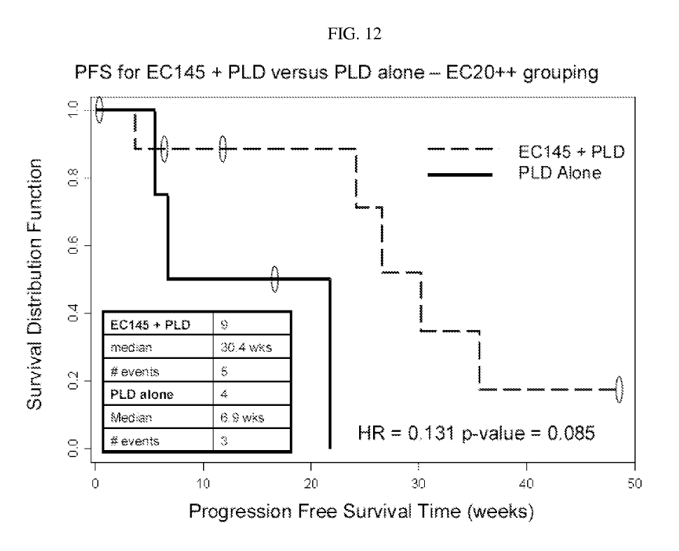

Kaplan-Meier curves for progression free survival time in the ongoing phase 2 trial in women with platinum-resistant ovarian cancer, at the time of the interim analysis, for subjects enrolled at sites with nuclear imaging capabilities who were scanned with EC20 prior to study treatment and scored as EC20 positive (EC20++ status) prior to study treatment (EC145 in combination with PLD versus PLD alone) are shown in FIG. 12.

Response to Therapy at the interim according to RECIST (version 1.0) is shown in the following table. Scan frequency and timing of assessments (every 6 weeks for 24 weeks, then every 8 weeks for the balance of the study participation) were equal for each arm.

| | | |
|---|---|---|
| RECIST _{v1.0} Confirmed | EC145/PLD | PLD |
| Response to Treatment | n = 54 | n = 17 |
| Complete Response (CR) | 0 (0.0%) | 0 (0.0%) |
| Partial Response (PR) | 9 (16.7%) | 4 (14.8%) |
| Stable Disease (SD) | 33 (61.1%) | 12 (44.4%) |
| Progressive Disease (PD) | 12 (22.2%) | 11 (40.7%) |
| Overall Disease Control | 42 (77.8%) | 16 (59.3%) |
| (SD + PR + CR) | | |

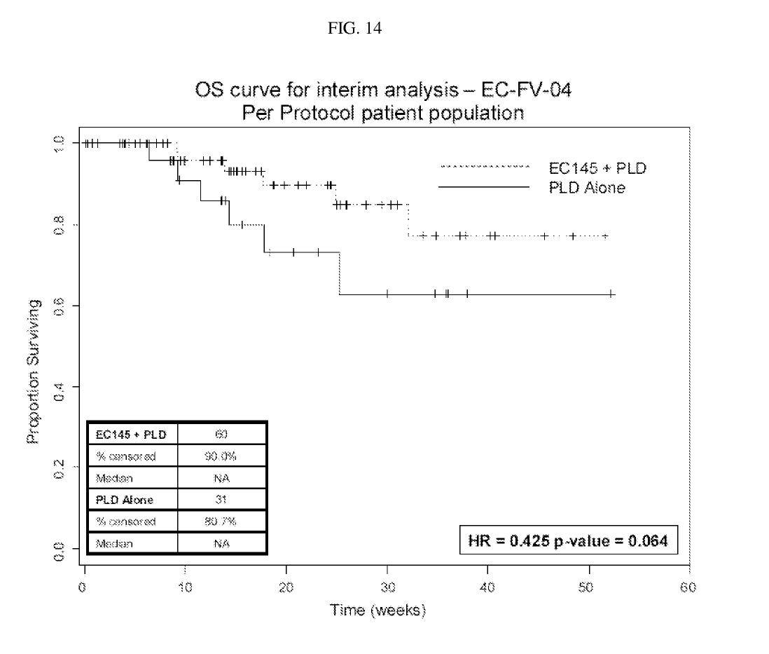

FIG. 14 shows a Kaplan-Meier graph of Overall Survival (OS) for patients treated with EC145 in combination with PLD versus those receiving PLD alone. At the time of the pre-specified interim analysis, median overall survival was trending toward statistical significance with hazard ratio of 0.425 (details on chart).

The protocol for this trial is summarized at http://www.clinicaltrials.gov/ct2/show/NCT00722592?term=platinum+resistant+ovarian+can cer&rank=2 which is incorporated by reference herein.

### Example 26

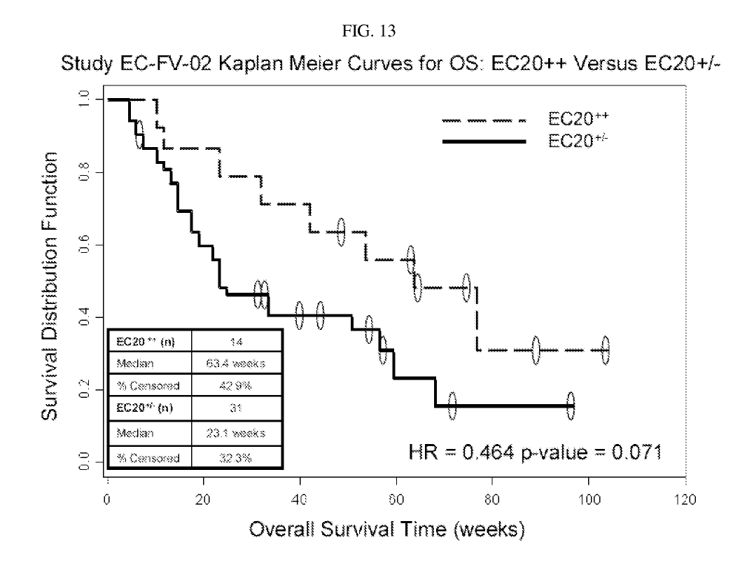

Kaplan-Meier curves for overall survival time in Study EC-FV-02, a trial in women with advanced ovarian and endometrial cancers who were scanned with EC20 prior to study treatment and assessed as EC20 positive (EC20++ status) compared to those assessed as EC20+ status or EC20− status prior to study treatment are shown in FIG. 13. This curve examines the utility of selecting patients who benefit from the single agent EC145 in highly refractory ovarian cancer patients.

### Example 27

EC20 Patient Scanning Procedure. Following completion of screening procedures and confirmation of eligibility, all subjects received one intravenous injection of 0.5 mg of folic acid followed within 1-3 minutes by a 1-2 mL injection of 0.1 mg of EC20 labeled with 20-25 mCi of technetium-99m. Patients then underwent SPECT imaging (mid-thigh to head, posterior and anterior images) 1-2 hours following injection of EC20. Target lesions were selected by the radiologist according to RECIST (v1.0) criteria. Subsequently, nuclear medicine physicians assessed EC20 uptake for each target lesion visually, and classified the uptake as “positive” (marked/mild uptake) or “negative” (no uptake).

### Example 28

EC20 Lesion Scoring Procedure. Lesions less than 1.5 cm in longest dimension (LD) were considered “non-evaluable” unless the nuclear medicine reader identified them as having unequivocal uptake of EC20, in which case they were characterized as “positive.” Since certain organs (ie, liver, spleen, bladder, and kidney) have an inherently high uptake of EC20, target lesions located in these organs were considered “non-evaluable.”

All evaluable lesions were categorized into two mutually exclusive groups: 1) EC20+ (uptake of EC20) and 2) EC20−(neg) (no uptake of EC20). Change in lesion size was compared between the 2 groups using analysis of variance (ANOVA). For each lesion, a treatment response was determined. Lesions with at least a 20% reduction in size were classified as responders (mPR), and those with at least a 20% increase in size were classified as progressive disease (PD). Lesions not meeting either the mPR or PD criteria were classified as stable (SD). The percentages of mPR, SD, and PD lesions were compared between the EC20+ and EC20−(neg) groups using Fisher's exact test. The quantitative percent change in tumor size was measured using the Pearson correlation coefficient.

### Example 29

EC20 Patient Scoring Procedure A subject score was calculated by dividing the total number of EC20+ lesions by the total number of lesions (evaluable and nonevaluable). Patients were categorized into three mutually exclusive groups:

Group 1: EC20++ (100% EC20-positive target lesions)

Group 2: EC20+ (1-99% EC20-positive target lesions)

Group 3: EC20−(neg) (0% or no EC20 positive target lesions).

For example, the “subject score” for a subject with one EC20+ lesion and two EC20−(neg) lesions (three target lesions in total) would be 33% (1 of 3 lesions positive), placing the subjects in the EC20+ group. A subject with all target lesions EC20 positive would be categorized as EC20++ (3 of 3 lesions are positive).

The best overall response was determined for each subject using RECIST v1.0 (Therasse, 2000). RECIST overall response rate (ORR) and disease control rates (CR/PR/SD) were calculated for each of the 3 populations. For these analyses, subjects who came off study prior to evaluation were considered non-responders. Overall survival was analyzed using Kaplan-Meier techniques and Cox proportional hazards models (Kaplan, 1958; Mantel, 1966). Due to sample size considerations, the EC20+ and EC20−(neg) were pooled for the survival analyses.

Sample sets for ORR and DCR included all evaluable subjects (intent to treat) as well as a subset of subjects who failed less than or equal to 3 prior lines of therapy. Due to a restriction in sample size, only intent to treat (ITT) is included for the survival analysis.

### Example 30

Patient Demographics. Forty-five ovarian cancer subjects were evaluated. Key demographic and disease characteristics are shown in the table below. Overall, subjects were highly pretreated prior to entering the EC-FV-02 study, with a median number of 4 prior chemotherapeutic regimens (range of 1 to 14 regimens). Eighty percent of subjects had tumor burden>5 cm in LDₛᵤₘ.

Demographics of Patients Treated with EC20: EC-FV-02

| Characteristic | N (%) |
|---|---|
| No. of subjects | 45 |
| Median age (years) | 62 |

ECOG Performance Status

| | |
|---|---|
| 0 | 17 (37.8%) |
| 1 | 24 (53.3%) |
| 2 | 4 (8.9%) |

Type of cancer, n (%)

| | |
|---|---|
| Endometrial | 4 (8.9%) |
| Ovarian | 34 (75.6%) |
| Peritoneal | 7 (15.6%) |

Disease burden

| | |
|---|---|
| >5 cm | 36 (80%) |
| ≦5 cm | 9 (20%) |

### Example 31

### Lesion Assessment

Forty-five protocol-eligible subjects with a total of 216 RECIST-defined target tumors (ie, “lesions”) were included in this retrospective analysis (table below). One-hundred and forty-five (145/216; 67%) of the lesions were considered EC20 “evaluable.” Of these, 111 (77%) had EC20 “positive” uptake. Of the 71 lesions considered “non-evaluable,” 45 lesions were present in organs with high background uptake; 15 lesions did not have sufficient SPECT data for interpretation; and 11 lesions that were smaller than 1.5 cm in size were coded with uptake as “none.”

As shown in the table below, 145 lesions were classified with unequivocally “positive” or “negative” uptake of EC20. These lesions were included in lesion analysis. One-hundred and eleven lesions were EC20+ and 34 lesions were EC20−(neg).

Evaluable and Non-Evaluable Lesions

| Lesion Grouping | N (%) |
|---|---|
| Total number of RECIST-defined lesions | 216 (100%) |
| Non-Evaluable Lesions | 71 (33%) |
| No SPECT data | 15 (21%) |
| Negative lesion < 1.5 cm in size | 11 (15%) |
| Lesions found in organs with high background | 45 (63%) |
| Liver | 32/45 |
| Spleen | 9/45 |
| Kidney | 4/45 |
| Evaluable Lesions | 145 (67%) |
| Positive (EC20+) | 111 (77%) |
| Negative (EC20-(neg)) | 34 (23%) |

Eighty-seven percent of the subjects had EC20 uptake observed visually in at least 1 target lesion. EC20−(neg) lesions were slightly larger in size than the EC20+ lesions (2.8 cm versus 2.4 cm, respectively [p=0.01]).

### Example 32

### EC20 Lesion Scoring Analysis

As shown in the table below, 59% (n=65) of the EC20+ lesions exhibited stable disease (SD) or modified partial response (PR) as compared to a 27% SD rate in the EC20−(neg) population. These findings indicate that EC20 uptake discriminates between lesions that exhibited a modified PR or SD (p=0.0022) after exposure to EC145 versus lesions that, at best, exhibit SD. All of the lesions in the modified PR group were EC20+.

Lesions Response to EC145 Therapy by EC20 Uptake

| | EC20+ | EC20− |
|---|---|---|
| Change in Lesion Size | n (%) | n (%) |
| Number of evaluable lesions | 111 (100%) | 34 (100%) |
| mPR | 11 (10%) | 0 (0%) |
| SD | 54 (49%) | 9 (27%) |
| PD | 46 (41%) | 25 (73%) |
| mPR + SD | 65 (59%) | 9 (27%) |

### Example 33

### EC20 Patient Scoring Analysis

The DCR for all evaluable subjects, regardless of EC20 status, was 42.2% (Table below). The DCR increased with EC20 positivity. EC20++ subjects had the highest DCR followed by EC20+ and EC20−(neg) subjects: 57%, 36% and 33%, respectively. The ORR across all subjects was 5%. Consistent with the DCR analysis, the ORR in the EC20++ subgroup was the highest at 14%, with the other 2 groups at 0%. In the subgroup of less heavily treated subjects that had failed ≦3 prior therapies, DCR for the EC20++ group was 86% versus 50% and 0% in the EC20+ and EC20−(neg) groups, respectively.

EC20 Percent Positive

| | | All Eligible Patients (ITT) | | | Failed ≦3Prior Therapies | | |
|---|---|---|---|---|---|---|---|
| | All | EC20++ | EC20+ | EC20− | EC20++ | EC20+ | EC20− |
| | Eligible | 100% | 1-99% | (neg) | 100% | 1-99% | (neg) |
| | Patients | Positive | Positive | 0% Positive | Positive | Positive | 0% Positive |
| | (n = 45) | (n = 14) | (n = 24) | (n = 7) | (n = 7) | (n = 8) | (n = 2) |
| CR/PR | 4% (2) | 7% (1) | 4% (1) | 0% (0) | 14% (1) | 13% (1) | 0% (0) |
| SD | 38% (16) | 50% (7) | 33% (8) | 29% (2) | 71% (5) | 37% (3) | 0% (0) |
| PD | 58% (26) | 43% (6) | 63% (15) | 71% (5) | 14% (1) | 50% (4). | 100% (2) |
| DCR | 42% (19) | 57% (8) | 36% (9) | 33% (2) | 86% (6) | 50% (4) | 0% (0) |

Results from this exploratory study of subjects treated with EC145 showed a trend for greater survival in the group with 100% positive lesions. The median overall survival in these subjects was 63.4 weeks versus 23.1 weeks in subjects with less than 100% positive tumors (hazard ratio=0.46, p=0.071).

### Example 34

### EC20: Chemistry Manufacturing and Controls

### Manufacture

EC20 is synthesized using standard Fmoc-based solid phase peptide synthesis chemistry as described above and in the diagram below. Starting with resin bound cysteine, protecting group removal is followed by coupling of the amino acid residue using standard reagents. After the last coupling step and deprotection, the peptide is cleaved from the resin. Crude product is precipitated and isolated by filtration. The purity of crude EC20 is about 90%.

Crude EC20 is purified by preparative column chromatography. EC20 is precipitated and isolated by filtration. The purity of the final drug substance is≧97%.

### Characterization

EC20 drug substance has been characterized by ¹H and ¹³C NMR analysis, by electrospray-mass spectroscopy, amino acid analysis, and peptide content. All methods confirmed the structure shown above.

### Process-Related Impurities

EC20 is purified by column chromatography to ensure that no starting materials or reagents used in the preparation of EC20 are present in the EC20 drug substance. Residual solvent levels are assessed by GC analysis of the isolated drug substance.

The specification for EC20 drug substance is shown in the table below.

| Attribute | Test Method | Limit | |
|---|---|---|---|
| Appearance | Visual Inspection | Yellow/off-white powder | |
| Identity | ESI-MS | M + H⁺ 746.2 ± 0.5 | |
| Purity | RP-HPLC | ≧95.0% | |
| Individual Specified | RP-HPLC | A | ≦1.0% |
| Impurities | | F | ≦1.0% |
| | | H | ≦1.0% |
| | | L | ≦1.0% |
| | | P1 | ≦1.5% |
| | | T | ≦1.0% |
| Individual Unspecified | RP-HPLC | ≦1.0% | |
| Impurities | | | |
| Total Impurities | RP-HPLC | ≦5.0% | |
| Peptide Content | % Nitrogen | ≧88.0% | |
| Moisture | Karl Fischer | ≦10.0% | |
| Endotoxin | USP <85> | <2.0 | EU/mg |
| Microbial Enumeration | USP <61> | <10 | CFU/100 mg |
| Methanol | Gas Chromatography | ≦0.5 | μg MeOH/mg API |
| Acetonitrile | GC | ≦1.0 | ACN/mg APIμg |
| Hydrazine | HPLC | <1.5 | microgram/mg API |

EC20 Drug Substance is stored at −20 ° C. in amber glass bottles with butyl rubber stoppers. Stability data show that drug substance is stable under these conditions for more than 24 months.

### Example 35

### Description and Composition of the Investigational Medicinal Drug Product

The medicinal product is a kit for the preparation of ^{99m}Tc-EC20. The product is a lyophilized, sterile, light yellow solid.

Quantity per vial of EC20 Drug Product

| Ingredient | Quantity per vial (mg) |
|---|---|
| EC20 Drug Substance | 0.100 |
| Sodium α-D-Glucoheptonate | 80 |
| Dihydrate(Glucoheptonate) | |
| Tin (II) Chloride Dihydrate | 0.080 |
| (SnCl₂•2H₂O) | |

The preparation of the final dosage form, ^{99m}Tc-EC20 for injection, is carried out at the clinical trial site, in accordance with standard practices for ^{99m}Tc-based diagnostic agents.

The EC20 drug product is a single use vial that contains all the components necessary to prepare an effective imaging agent by the addition of sterile sodium pertechnetate. The drug substance is formulated with Tin (II) chloride and sodium-α-D-glucoheptonate in the amounts shown in the table below. This formulation is typical for agents that utilise metastable technetium as a source of radioactivity. The EC20 drug product chelates technetium, and when an aqueous solution of freshly prepared metastable technetium is used to reconstitute the EC20, the technetium is chelated and the imaging agent is formed.

EC20 DP components

| | Mass or | | | Concentration after | |
|---|---|---|---|---|---|
| Component | Activity | | Mol | reconstitution | Molar Ratio |
| EC20 | 100 | μg | 1.3 × 10⁻⁷ | 1.3 × 10⁻⁴M | 255 |
| Sodium α-D- | 80 | mg | 2.8 × 10⁻⁴ | 0.28M | 550,000 |
| glucoheptonate | | | | | |
| Tin (II) chloride | 80 | μg | 3.5 × 10⁻⁷ | 3.5 × 10⁻⁴M | 686 |
| ^{99m}Tc/⁹⁹Tc(1:4) | 30 | mCi | 5.1 × 10⁻¹⁰ | 5.1 × 10⁻⁷M | 1 |

### Batch Formula

The batch formula is provided in the table below for the typical 2000 vial batch of EC20 Drug Product:

EC20 Batch Formula

| Ingredient | Quantity | |
|---|---|---|
| EC20 Drug Substance | 0.20 | g |
| Sodium α-D-Glucoheptonate Dihydrate | 160 | g |
| (Glucoheptonate) | | |
| Tin (II) Chloride Dihydrate (SnCl₂•2H₂O) | 0.16 | g |
| Sterile Water For Injection (SWFI) | 5 | L |
| Nitrogen (inert atmosphere) | As needed | |
| Hydrochloric acid | 4.3 | mL |
| Sodium Hydroxide | 10 | g |

### EC20 DP Fill Process

The manufacturing process is performed under a nitrogen or argon atmosphere.

Preparation of Glucoheptonate solution: The empty formulation vessel is weighed with a suitable stir bar. Deoxygenated SWFI is added to the pre-weighted formulation vessel. Glucoheptonate is added to the formulation vessel using a glass funnel. The weighing container and funnel are rinsed with deoxygenated SWFI and the rinses added to the formulation solution.
Preparation SnCl₂.2H₂O solution: The SnCl₂.2H₂O is weighed into an appropriately sized flask. The SnCl₂.2H₂O is dissolved in deoxygenated 0.2M HCl.
Preparation of the EC20 solution: The SnCl₂.2H₂O solution is slowly transferred to the prepared Glucoheptonate solution, with continuous stirring. The appropriate amount of EC20 (calculated from the known peptide content) is transferred to the Glucoheptonate/SnCl₂ solution. 1.0M NaOH and/or 0.2M HCl is slowly added until the pH reaches 6.8±0.2. The solution is diluted to the desired target weight±0.25% with deoxygenated SWFI and stirred for a minimum of 5 minutes. A pre-filtration bioburden sample is drawn from the formulation vessel using aseptic technique and placed into a sterile container with sterile cap closure. The filtration apparatus, two sterile filters in series, and receiving vessel are set up and the EC20 formulation solution is filtered into an appropriate receiving vessel through the 0.22 micron, sterile filter using a peristaltic pump. A post-filtration filter integrity test is performed. If the recorded pressure fails, the test is repeated one time. If it fails a second time, new filters may be installed and the process can be repeated.
Filling and Stoppering: Filling and stoppering is carried out aseptically in a Class 100 filling area. All containers, vessels, mixing devices and utensils which contact the product or materials going into the product are properly cleaned and sterilized or depyrogenated. Set-up and fill checks are performed gravimetrically based on calculated density. The vials are filled and stoppered. Stoppers are placed in the lyophilisation position (half seated) before the vials are removed from the work station. Lyophilizer trays are loaded into the chamber onto shelves and then chilled to −45° C.±3° C. Once the product is frozen, the vacuum pump evacuates the chamber. The drying cycle is terminated manually by closing the vacuum pump valve after holding a shelve temperature of 30° C.-35° C. for≧10 hours. The shelf stoppering mechanism is activated after purging the chamber to 7-10 mmHg with filtered nitrogen. When all vials are stoppered the chamber is back filled with filtered nitrogen to atmospheric pressure and the product trays are removed from the chamber and capped with aluminum seals. Vials are labelled after capping and stored at −20° C.±5° C.

EC20 Drug Product Excipients Specifications

| Ingredient | Specification | Limit | Result | |
|---|---|---|---|---|
| Sterile Water for | USP | Not applicable | Not applicable | |
| Injection | | | | |
| Tin (II) Chloride | USP | Not applicable | Not applicable | |
| Dihydrate (SnCl₂•2H₂O) | | | | |
| Sodium α-D- | Assay, HPLC | 98-102% | 100% | |
| Glucoheptonate Dihydrate | Solution | 10% Aqueous | Pass | |
| (Glucoheptonate) | | solution is clear | | |
| | | and colorless | | |
| | | 6.5-9.0 | | |
| | pH | ≦12.8% | 8.7 | |
| | LOD, TGA | <200 CFU/gm | 8.0% | |
| | Bioburden^{a} | <1100 EU/gm | <10 | CFU/gm |
| | Endotoxin^{a} | | <1.2 | EU/gm |

^{a}Bioburden and Endotoxin were determined prior to using the glucoheptonate as an excipient

All other assay results were obtained from the vendors' certificate of analysis.

### Example 36

### Typical Conversion of Vinblastine Sulfate into Desacetylvinblastine Hydrazide

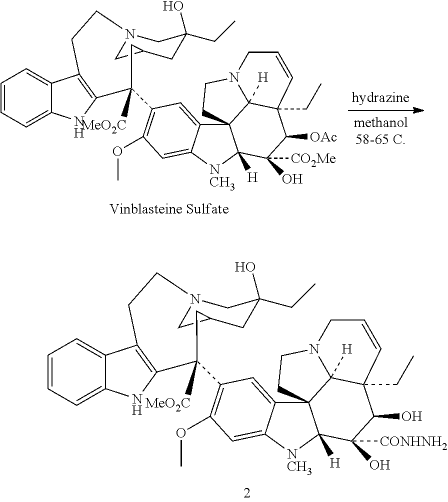

### Materials

Vinblastine Sulfate: USP; FW=909.05 g/mole; Methanol: anhydrous; Hydrazine: anhydrous; FW=32 g/mol; De-ionized water; Ethyl acetate: LC/GC grade; Toluene: LC/GC grade; Monobasic sodium phosphate: ≧99.0%; FW=120 g/mole; Dibasic sodium phosphate: ≧99.0%; FW=142 g/mole; Sodium chloride: reagent grade; FW=58.4 g/mole; Sodium sulfate: anhydrous; 5-norbornen-2-carboxylic acid.

### Procedure

The reaction, extractive work-up and isolation are run under a nitrogen or argon atmosphere. Pressure filters are used to remove the sodium sulfate and capture the product. The sodium chloride solutions used in the quench and wash are sparged with nitrogen or argon until the dissolved oxygen level is not more than 0.9 ppm.

Vinblastine sulfate and anhydrous methanol are charged to an argon purged reactor. 5-Norbornene-2-carboxylic acid and anhydrous hydrazine are added to the reactor. The mixture is stirred, and after the solids dissolve, heat the mixture to around 60° C. By HPLC analysis, when the reaction is complete, it is cooled, quenched and extracted into ethyl acetate. After drying, the product is crystallized from ethyl acetate and toluene. The solids are dried under vacuum overnight at room temperature.

The buffered NaCl contains: 10.0 g NaCl, 7.10-7.30 g NaH₂PO₄, 4.40-4.60 g of Na₂HPO₄ and 90 mL of water. The solution is sparged with argon or nitrogen (dissolved oxygen content<0.9 ppm).

The typical isolated yield is 50-60% of the theoretical maximum.

### Example 36

### Steps 2 and 3 of the EC145 Process

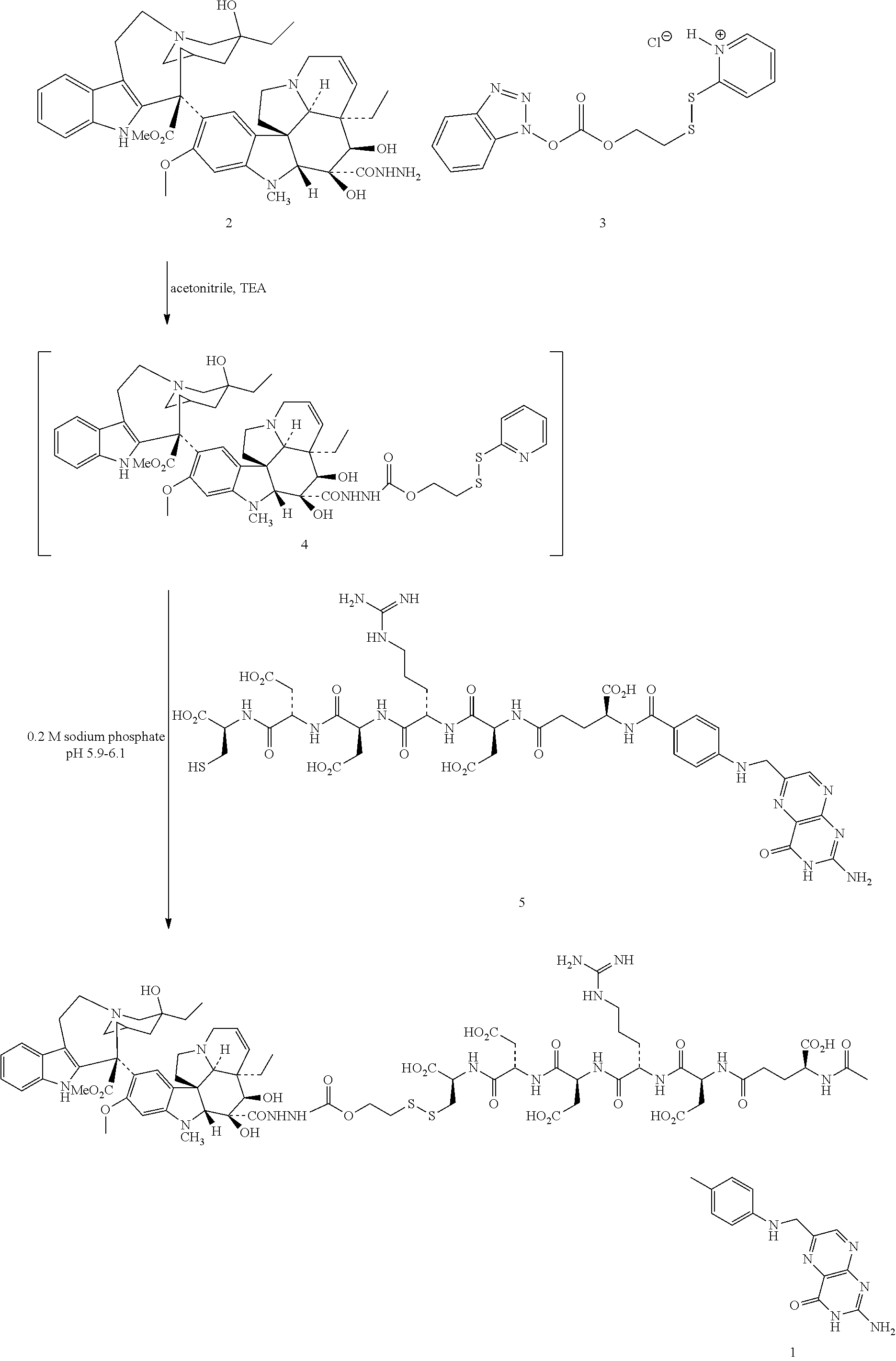

### Step 2 and Step 3 Processes

### Materials

Desacetylvinblastine hydrazide: FW=768.9 g/mol; 20.5 g, 26.7 mmol; Mixed Carbonate (3): FW=384.9 g/mol; 10.7 g, 27.8 mmol; Acetonitrile: q.s.: Triethylamine: FW=101.2 g/mol; 2.67 g, 26.4 mmol; Na₂PO₄ 7 H₂O: 47.84 g; EC119: 29.9 g 28.6 mmole; 0.5 N HCl: q.s.; WFI: q.s.

### Procedure

Note that all of the water used in this process is WFI.

Purge an appropriate vessel with Argon. Charge 20.5±0.3 g of des-acetylvinblastine hydrazide; this charge is potency adjusted, i.e., if the potency were 90.0%, the charge would be 22.8 g. Charge 10.7±0.2 g of Mixed Carbonate (potency adjusted). Charge 800±30 mL of acetonitrile and 2.67±0.11g of triethylamine. Mix under Argon at 10-14° C. for 20-28 hours. Take a sample for HPLC (EC145-CMC-AM-0001, version 2.3). The expected result is the ratio of CDSI to hydrazide≧25:1. If not, continue mixing under Argon at 10-14° C. for 2-4 hours and sample again.

Sparge 780-820 mL of water with Argon until the dissolved oxygen level is less than 0.9 ppm; record dissolved oxygen level. Dissolve 47.8±0.5 g of sodium phosphate dibasic heptahydrate in the deoxygenated water. To a suitable container, add 29.8±0.5 g of EC119; (charge is potency adjusted). Add the sodium phosphate solution to the EC119 and mix under Argon. Measure the solution's pH and adjust the pH to 5.8-6.2 with 0.5 N HCl if necessary.

Add the buffered EC119 solution to the reaction mixture. Mix under Argon at 20-25° C. for 60-75 minutes. Take a sample for HPLC (EC145-CMC-AM-0001, version 2.3). If the ratio of EC145 to CDSI ≧25:1, proceed. If not, continue mixing under Argon at 20-25° C. and sample again. If the ratio of EC145 to CDSI≧25:1, proceed. If not, add an additional 1 g of EC119 and mix under Argon at 20-25° C. for 30 minutes and sample again.

Prepare 6.9 L-7.1 L of 25 mM phosphate buffer, 185-195 mM NaCl, pH 7.2-7.5 made from water sparged with Argon until the dissolved oxygen level is less than 0.9 ppm. Dilute the reaction mixture with this buffer. If the mixture develops more than a faint haze, the product solution needs to be filtered (Whatman Polycap TC75 or TC150, 0.45 or 1.0 micron); this filtration may be done while loading the product onto the Biotage column.

### Liquid Chromatographic Purification

Use a Biotage 150M, C18 cartridge. This size cartridge can accommodate a reaction mixture twice the size of the one currently described.

Column preparation:

a. Flush the column with

i. 12-13 L of acetonitrile

ii. 12-13 L of 80% acetonitrile and 20% water (v/v)

iii. 12-13 L of 50% acetonitrile and 50% water (v/v)

iv. 12-13 L of 10% acetonitrile and 90% water (v/v)

Purification:

Prepare a 25 mM phosphate buffer, (185-195 mmol) NaCl, pH 7.3-7.5
Sparge the buffer with argon until the dissolved oxygen content is≦0.9 ppm.
Prepare: 41 L of 10% acetonitrile in buffered saline (v/v); 13 L of 16% acetonitrile in buffered saline (v/v), 52 L of 27% acetonitrile in buffered saline (v/v).
Check the dissolved oxygen content of the mobile phase solutions. If the dissolved oxygen content is greater than 0.9 ppm, sparge the mobile phase with argon or nitrogen until the dissolved oxygen level is≦0.9 ppm.
Flush the column with 26-27 L of the 10% acetonitrile mobile phase.
Load the product solution onto the column
Elute the product using the following sequence of mobile phases:

i. 13-14 L of the 10% acetonitrile mobile phase.

ii. 13 L of the 16% acetonitrile mobile phase.

iii. 51-52 L of the 27% acetonitrile mobile phase.

Notes: An inline uv detector is helpful; Product should come out starting at 15-19 L of the 27% acetonitrile mobile phase with a bandwidth of 8-13 L.

### Fraction Evaluation

i. HPLC Method EC145-CMC-IP-0001

ii. Passing fraction=≧97.0% EC145 and no impurity ≧0.8%

Post-Run Column Treatment:

The column can be reused once. If the column will be used for a second run, perform ii-iv.

i. Flush column with 12-13 L of 1:1 acetonitrile-water.

ii. Flush column with 20-22 L of acetonitrile

iii. Repeat column preparation steps ii-iv

### Ultra-Filtration

Sparge q.s. water with argon or nitrogen until the dissolved oxygen level is less than 0.9 ppm. Passing chromatography fractions are combined and diluted with an equivalent volume of sparged water. Assemble an ultra-filtration apparatus using a Millipore regenerated cellulose membrane with nominal MW cutoff of 1000 (cat# CDUF002LA) and rinse it with 9 L of deoxygenated water. Start ultra-filtration of the product solution. Maintain a backpressure of 30-50 psi. Continue ultra-filtration until the retentate volume is 2 to 3 L. Add 11 to 12 L of deoxygenated water. Continue ultra-filtration until the retentate volume is 2 to 3 L. Add 11 to 12 L of deoxygenated water. Continue ultra-filtration until the retentate volume is 2 to 3 L. Add 8 to 10 L of deoxygenated water. Continue the ultra-filtration until the retentate volume is 2 L. The ultra-filtration endpoint must be determined by analyzing a sample of the retentate via GC and concentration. The specification is≦50 micrograms of acetonitrile per milligram of EC145. If not achieved, perform another cycle of the ultra-filtration.

The API solution's concentration must be adjusted so that the packaged material is 6 to 12 mg/mL. At the completion of the ultra-filtration, the apparatus will be rinsed with 1 liter of water. Therefore, continue ultra-filtration or add water as necessary. Once the product solution is out of the ultra-filtration apparatus, rinse the ultra-filtration apparatus with 1 L of deoxygenated water and combine with the product solution.

After the rinse is combined with the product solution, this solution must be filtered through a 0.2 micron absolute filter, and this filtrate is packaged (performed under an inert atmosphere).

The yield of isolated product is 50-60% of the theoretical maximum.

1. A method of determining whether EC145 is indicated for the treatment of a patient with an ovarian tumor or a lung tumor, the method comprising the step of
determining whether functionally active folate receptors are present on the tumor of the patient wherein the EC145 is indicated for the treatment of the patient with the tumor if functionally active folate receptors are present on the tumor.

2. The method of claim 1 further comprising the step of administering to the patient EC20 for detection of the functionally active folate receptors.

3. The method of claim 2 further comprising the step of administering to the patient an unlabeled folate prior to administration of the EC20.

4. The method of claim 2 wherein the EC145 is indicated for the treatment of the patient with the tumor if the radioactive signal produced by the EC20 upon binding to the tumor compared to the background radioactive signal produced by the EC20 is indicative of a clinical benefit to the patient.

5. The method of claim 4 wherein the clinical benefit is progression-free survival of the patient.

6. The method of claim 4 wherein the clinical benefit is inhibition of tumor growth.

7. The method of claim 4 wherein the clinical benefit is selected from the group consisting stable disease, a partial response, and a complete response.

8. The method of claim 4 wherein the level of expression of the functionally active folate receptors is quantified based on a tumor to background ratio of the radioactive signal produced by the EC20 to the background radioactive signal.

9. The method of claim 8 wherein the tumor to background ratio is at least about 1.2.

10. The method of claim 8 wherein the tumor to background ratio is at least about 1.3.

11. The method of claim 8 wherein the tumor to background ratio is at least about 1.4.

12. The method of claim 4 wherein the tumor is an ovarian tumor.

13. The method of claim 12 wherein the tumor is a platinum-resistant ovarian tumor.

14. The method of claim 4 wherein the tumor is a lung tumor.

15. The method of claim 14 wherein the tumor is a non-small cell carcinoma of the lung.

16. The method of claim 4 wherein either the EC145, the EC20, or both are in a parenteral dosage form.

17. The method of claim 16 wherein the dosage form is selected from the group consisting of intradermal, subcutaneous, intramuscular, intraperitoneal, intravenous, and intrathecal.

18. The method of claim 17 wherein the EC145 is in a composition and wherein the composition further comprises a pharmaceutically acceptable carrier.

19. The method of claim 4 wherein the composition comprising the EC20 further comprises a pharmaceutically acceptable carrier.

20. The method of claim 4 wherein the EC145 is administered in a therapeutically effective amount.

21. The method of claim 4 wherein the EC20 is administered in a therapeutically effective amount.

22. The method of claim 1 further comprising the step of administering to the patient doxorubicin.

23. The method of claim 22 wherein the doxorubicin is in the form of a pegylated liposomal doxorubicin.

24. A method of determining whether EC145 is indicated for the treatment of a patient with an ovarian tumor or a lung tumor, the method comprising the step of
administering to the patient a composition comprising EC20,
wherein the EC145 is indicated for the treatment of the patient with the tumor if the tumor of the patient has functionally active folate receptors wherein the functionally active folate receptors are capable of detection with EC20.

25. The method of claim 24 further comprising the step of administering to the patient an unlabeled folate prior to administration of the EC20.

26. The method claim 24 or further comprising the step of administering to the patient doxorubicin.

27. The method of claim 26 wherein the doxorubicin is in the form of a pegylated liposomal doxorubicin.

28. A method of predicting a response of an ovarian tumor or a lung tumor of a patient to therapy with EC145, the method comprising the steps of
a) administering to the patient EC20 wherein the EC20 produces a radioactive signal;
b) quantifying the radioactive signal produced by the EC20 upon binding of the EC20 to the tumor;
c) quantifying the background radioactive signal produced by the EC20;
d) comparing the radioactive signal produced upon binding of the EC20 to the tumor to the background radioactive signal; and
e) predicting the response of the tumor to the therapy based on the comparison.

29. The method of any one of claims 1, 24 and 28 wherein 15 mg/month of the EC145 is administered.

30. A method of treatment of platinum-resistant ovarian cancer in a patient in need thereof comprising administering a therapeutic amount of EC145 in combination with a therapeutic amount of pegylated liposomal doxorubicin.

31. (canceled)

32. (canceled)

33. A method of obtaining a clinical benefit compared to treatment with a therapeutic amount of pegylated liposomal doxorubicin in the treatment of platinum-resistant ovarian cancer in a patient in need thereof comprising administering a a therapeutic amount of EC145 in combination with a therapeutic amount of pegylated liposomal doxorubicin.

34. The method of claim 33 wherein the clinical benefit is progression-free survival.

35. The method of claim 33 wherein the clinical benefit is overall survival.

36. The method of claim 30 or 33 wherein the purity of EC145 is at least 90%.

37. The method of claim 30 or 33 wherein the EC145 is provided in an aqueous sterile liquid formulation the components of which comprise monobasic sodium phosphate monohydrate, dibasic disodium phosphate dihydrate, sodium chloride, potassium chloride and water for injection.

38. The method of claim 30 or 33 wherein the treatment further comprises a bowel regimen.

39. The method of claim 30 or 33 wherein the EC145 is administered as a bolus over about 10 to 20 seconds.

40. The method of claim 30 or 33 further comprising administering EC20 to the patient prior to treatment and assessing the patient to have EC20++ status.

41. A method of selecting a patient for treatment as described in claim 30 or 33 comprising administering EC20 to the patient prior to treatment and assessing the patient to have EC20++ status.

42. A pharmaceutical composition comprising EC145 in an aqueous sterile liquid formulation the components of which comprise monobasic sodium phosphate monohydrate, dibasic disodium phosphate dihydrate, sodium chloride, potassium chloride and water for injection.

43. A dosage unit comprising EC145 drug product for intravenous administration as 2.0 mL of an aqueous sterile liquid formulation, pH 7.4, which dosage unit contains 1.4 mg/mL of EC145.

44. The dosage unit of claim 43 which is an ampoule, a sealed vial or a prefilled syringe.

45. The dosage unit of claim 44 which is a sealed vial.

46. A method of determining whether a patient with a tumor has functionally active folate receptors present on the tumor of the patient, the method comprising the step of administering an effective amount of EC20 to the patient for detection of the functionally active folate receptors.

47. The method of claim 46 wherein the tumor is an ovarian tumor or a lung tumor.

48. The method of claims 46 wherein the tumor is a primary tumor or a metastatic tumor.

49. The method of any one of claim 1, 24 and 46 wherein the functionally active folate receptors are detected visually.

50. The method of claim 49 wherein the visual detection of functionally active folate receptors is used to determine folate receptor status of the patient.

51. The method of claim 50 wherein the folate receptor status of the patient is selected from the group consisting of EC20++, EC20+, and EC20−.

52. The method of claim 51 wherein the folate receptor status is EC20++.

53. The method of claim 52 wherein treatment with EC145 is indicated.

54. The method of claim 52 wherein EC20++ status correlates with a clinical benefit to the patient.

55. The method of claim 54 wherein the clinical benefit is disease control rate.

56. The method of claim 54 wherein the clinical benefit is overall disease response rate.

57. The method of claim 54 wherein the clinical benefit is overall survival.

58. A method of treatment of a folate receptor expressing epithelial tumor in a patient in need thereof comprising administering a therapeutic amount of EC145 in combination with a therapeutic amount of doxorubicin.

59. (canceled)

60. (canceled)

61. A method of achieving a clinical benefit in the treatment of a folate receptor expressing epithelial tumor in a patient in need thereof comprising administering a therapeutic amount of EC145 in combination with a therapeutic amount of pegylated liposomal doxorubicin.

62. The method of claim 61 in which the clinical benefit is progression-free survival.

63. The method of claim 61 in which the clinical benefit is overall survival.

64. The method of claim 58 wherein the doxorubicin is in the form of a pegylated liposomal doxorubicin.

65. The method of claim 58 wherein the folate receptor expressing epithelial tumor is an ovarian, endometrial or non-small cell lung cancer (NSCLC) tumor.

66. The method of claim 65 wherein the folate receptor expressing epithelial tumor is an ovarian tumor.

67. The method of claim 64 wherein the folate receptor expressing epithelial tumor is an ovarian, endometrial or non-small cell lung cancer (NSCLC) tumor.

68. The method of claim 67 wherein the folate receptor expressing epithelial tumor is an ovarian tumor.

69. A method of treating platinum-resistant ovarian cancer or lung cancer, the method comprising the step of
administering EC145 wherein the EC145 is for use in combination with pegylated liposomal doxorubicin, and is indicated for the treatment of tumors which comprise platinum-resistant ovarian cancer or lung cancer, and wherein the treatment is indicated if at least one tumor is folate receptor positive.

70. The method of claim 69 wherein the treatment is indicated for lung cancer.

71. The method of claim 70 wherein the lung cancer is non-small cell lung cancer.

72. The method of claim 69 wherein the treatment is indicated for platinum-resistant ovarian cancer.

73. The method of claim 72 wherein the ovarian cancer comprises one or more tumors and the tumor folate receptor positive status is EC20+ or EC20++.

74. The method of claim 73 wherein the tumor status is EC20+.

75. The method of claim 73 wherein the tumor status is EC20++.

76. The method of claim 73 wherein EC20++ denotes that about 100 percent of the ovarian tumors are folate-receptor positive and EC20+ denotes that about 1 percent to about 99 percent of the ovarian tumors are folate-receptor positive.

77. The method of claim 73 wherein the tumors are evaluated visually for folate receptor status.

78. The method of claim 72 further comprising the step of administering ^{99m}Tc-EC20 prior to administering EC145 to the patient.

79. A method of treating an ovarian cancer patient with positive folate receptor status or a lung cancer patient with positive folate receptor status, the method comprising the step of administering EC145 to the ovarian cancer patient with positive folate receptor status or to the lung cancer patient with positive folate receptor status.

80. The method of claim 79 wherein the cancer is lung cancer.

81. The method of claim 80 wherein the lung cancer is non-small cell lung cancer.

82. The method of claim 79 wherein the cancer is ovarian cancer.

83. The method of claim 82 wherein the cancer comprises one or more ovarian tumors and the tumor status is EC20+ or EC20++.

84. The method of claim 83 wherein the tumor status is EC20+.

85. The method of claim 83 wherein the tumor status is EC20++.

86. The method of claim 83 wherein EC20++ denotes that about 100 percent of the ovarian tumors are folate-receptor positive and EC20+ denotes that about 1 percent to about 99 percent of the ovarian tumors are folate-receptor positive.

87. The method of claim 83 wherein the tumors are evaluated visually for folate receptor status.

88. The method of claim 82 further comprising the step of 99m administering Tc-EC20 to the patient prior to administering EC145 to the patient.

89. The method of claim 79, 82, 83, 84, 85, 86, 87, or 88 further comprising the step of administering pegylated liposomal doxorubicin to the patient.

90. A method of treating ovarian cancer or a lung cancer with EC145, the method comprising the steps of
receiving results from imaging that show positive folate receptor status of at least one or more tumors; and
administering EC145.

91. The method of claim 90 wherein the cancer is lung cancer.

92. The method of claim 91 wherein the lung cancer is non-small cell lung cancer.

93. The method of claim 90 wherein the cancer is ovarian cancer.

94. The method of claim 93 wherein the folate receptor status of the tumors is EC20+ or EC20++.

95. The method of claim 94 wherein the folate receptor status of the tumors status is EC20+.

96. The method of claim 94 wherein the folate receptor status of the tumors is EC20++.

97. The method of claim 94 wherein EC20++ denotes that about 100 percent of the ovarian tumors are folate-receptor positive and EC20+ denotes that about 1 percent to about 99 percent of the ovarian tumors are folate-receptor positive.

98. The method of claim 94 wherein the tumors are evaluated visually for folate receptor status.

99. The method of claim 90 wherein the results of the imaging are obtained by administering ^{99m}Tc-EC20.

100. The method of claim 90, 93, 94, 95, 96, 97, 98, or 99 further comprising the step of administering pegylated liposomal doxorubicin to the patient.

101. A method of treating a ^{99m}Tc-EC20 imaged patient having (a) ovarian cancer and one or more ovarian tumors or (b) lung cancer and one or more lung tumors wherein EC145 is used for treatment of an EC20+ or an EC20++ patient, the method comprising the step of
administering EC145 to the patient.

102. The method of claim 101 wherein the patient has lung cancer.

103. The method of claim 102 wherein the lung cancer is non-small cell lung cancer.

104. The method of claim 101 wherein the patient has ovarian cancer.

105. The method of claim 101 wherein the status is EC20+.

106. The method of claim 101 wherein the status is EC20++.

107. The method of claim 101 wherein EC20++ denotes that about 100 percent of the ovarian tumors are folate-receptor positive and EC20+ denotes that about 1 percent to about 99 percent of the ovarian tumors are folate-receptor positive.

108. The method of claim 101 wherein the tumors are evaluated visually for folate receptor status.

109. The method of claim 101, 104, 105, 106, 107, or 108 further comprising the step of administering ^{99m}Tc-EC20 to the patient prior to administering EC145 to the patient.

110. The method of claim 109 further comprising the step of administering pegylated liposomal doxorubicin to the patient.

111. A method for determining that EC145 is indicated for the treatment of an imaged patient having (a) ovarian cancer and one or more ovarian tumors or (b) lung cancer and one or more lung tumors, the method comprising the steps of
administering a radiolabelled folate-targeted conjugate to the patient;
generating an image of the patient; and
using the image to visually determine folate receptor status of the patient.

112. The method of claim 111 wherein the patient has lung cancer.

113. The method of claim 112 wherein the lung cancer is non-small cell lung cancer.

114. The method of claim 111 wherein the patient has ovarian cancer.

115. The method of claim 114 wherein the cancer comprises one or more ovarian tumors and the tumor status is EC20+ or EC20++.

116. The method of claim 115 wherein the tumor status is EC20+.

117. The method of claim 115 wherein the tumor status is EC20++.

118. The method of claim 115 wherein EC20++ denotes that about 100 percent of the ovarian tumors are folate-receptor positive and EC20+ denotes that about 1 percent to about 99 percent of the ovarian tumors are folate-receptor positive.

119. The method of claim 111, 112, 113, 114, 115, 116, 117, or 118 further comprising the step of administering EC145 to the patient.

120. The method of claim 118 wherein the radiolabelled folate-targeted conjugate is ^{99m}Tc-EC20.

121. The method of claim 52 further comprising the step of administering pegylated liposomal doxorubicin to the patient.

122. A method of assessing whether EC145 treatment is indicated for a patient with ovarian cancer or lung cancer, the method comprising the steps of
administering ^{99m}Tc-EC20 to the patient; and
imaging one or more ovarian cancer metastatic lesions of the patient or one or more lung cancer lesions of the patient by generating an image wherein the image is for determination of folate receptor status of the ovarian or lung cancer metastatic lesions of the patient.

123. The method of claim 122 wherein the patient has lung cancer.

124. The method of claim 123 wherein the lung cancer is non-small cell lung cancer.

125. The method of claim 122 wherein the patient has ovarian cancer.

126. The method of claim 125 wherein the ^{99m}Tc-EC20 is administered to the patient after radiolabelling the ^{99m}Tc-EC20 with ^{99m}Tc using a sodium pertechnetate solution.

127. The method of claim 126 wherein the cancer comprises one or more ovarian lesions and the lesion status is EC20+ or EC20++.

128. The method of claim 127 wherein the lesion status is EC20+.

129. The method of claim 127 wherein the lesion status is EC20++.

130. The method of claim 127 wherein EC20++ denotes that about 100 percent of the lesions are folate-receptor positive and EC20+ denotes that about 1 percent to about 99 percent of the lesions are folate-receptor positive.

131. The method of claim 127 wherein the tumors are evaluated visually for folate receptor status.

132. The method of claim 122, 123, 124, 125, 126, 127, 128, 129, 130, or 131 further comprising the step of administering EC145 to the patient after administering the ^{99m}Tc-EC20 to the patient.

133. The method of claim 132 further comprising the step of administering pegylated liposomal doxorubicin to the patient.

134. A method of treating an ovarian cancer patient or a lung cancer patient with EC145, the method comprising the steps of
administering folic acid to the patient;
administering ^{99m}Tc-EC20 to the patient;
imaging one or more ovarian tumors or one or more lung tumors of the patient wherein the image shows ^{99m}Tc-EC20 binding to the tumors and wherein the image is for determination of folate receptor status of the patient;
determining the folate receptor status of the patient using the image; and
administering EC145 to the patient if the folate receptor status is positive.

135. The method of claim 134 wherein the cancer is lung cancer.

136. The method of claim 135 wherein the lung cancer is non-small cell lung cancer.

137. The method of claim 134 wherein the cancer is ovarian cancer.

138. The method of claim 137 wherein the cancer comprises one or more ovarian tumors and the tumor status is EC20+ or EC20++.

139. The method of claim 138 wherein the tumor status is EC20+.

140. The method of claim 138 wherein the tumor status is EC20++.

141. The method of claim 138 wherein EC20++ denotes that about 100 percent of the ovarian tumors are folate-receptor positive and EC20+ denotes that about 1 percent to about 99 percent of the ovarian tumors are folate-receptor positive.

142. The method of claim 138 wherein the tumors are evaluated visually for folate receptor status.

143. The method of claim 134, 135, 136, 137, 138, 139, 140, 141, or 142 further comprising the step of administering pegylated liposomal doxorubicin to the patient.

144. A method for determining if treatment of an ovarian cancer patient or a lung cancer patient with EC145 is indicated, the method comprising the steps of
using a ^{99m}Tc-EC20 image to determine folate receptor status of the ovarian cancer or the lung cancer; and
administering EC145 to the patient if treatment with EC145 is indicated.

145. The method of claim 144 wherein the cancer is lung cancer.

146. The method of claim 145 wherein the lung cancer is non-small cell lung cancer.

147. The method of claim 144 wherein the cancer is ovarian cancer.

148. The method of claim 147 wherein the cancer comprises one or more ovarian tumors and the tumor status is EC20+ or EC20++.

149. The method of claim 148 wherein the tumor status is EC20+.

150. The method of claim 148 wherein the tumor status is EC20++.

151. The method of claim 148 wherein EC20++ denotes that about 100 percent of the ovarian tumors are folate-receptor positive and EC20+ denotes that about 1 percent to about 99 percent of the ovarian tumors are folate-receptor positive.

152. The method of claim 148 wherein the tumors are evaluated visually for folate receptor status.

153. The method of claim 144, 145, 146, 147, 148, 149, 150, 151, or 152 further comprising the step of administering pegylated liposomal doxorubicin to the patient.

154. A method of treating an ovarian cancer patient or a lung cancer patient with EC145, the method comprising the steps of
administering folic acid to the patient;
administering ^{99m}Tc-EC20 to the patient;
scintigraphically imaging one or more ovarian tumors or one or more lung tumors of the patient wherein the image shows ^{99m}Tc-EC20 binding to the tumors and wherein the image is for determination of the folate receptor status of the patient; and
administering EC145 to the patient if the folate receptor status is positive.

155. The method of claim 154 wherein the patient has lung cancer.

156. The method of claim 155 wherein the lung cancer is non-small cell lung cancer.

157. The method of claim 154 wherein the patient has ovarian cancer.

158. The method of claim 157 wherein ovarian cancer comprises one or more ovarian tumors and the tumor status is EC20+ or EC20++.

159. The method of claim 158 wherein the tumor status is EC20+.

160. The method of claim 158 wherein the tumor status is EC20++.

161. The method of claim 158 wherein EC20++ denotes that about 100 percent of the ovarian tumors are folate-receptor positive and EC20+ denotes that about 1 percent to about 99 percent of the ovarian tumors are folate-receptor positive.

162. The method of claim 158 wherein the tumors are evaluated visually for folate receptor status.

163. The method of claim 154, 155, 156, 157, 158, 159, 160, 161, or 162 further comprising the step of administering pegylated liposomal doxorubicin to the patient.
